SEQUENCE LISTING (95 sequences)

SEQ ID NO: 1 - DNA, 1139 bp, Rattus sp.
  CDS (33)..(494)
caggtctagagttggaatcgaagcctcttaaaatggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaagcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaataactggcttccagggtggcggtggtggcagcagtgatccatgagcctacagaggcccctcccccagctctggctgggcccttggctggactcctatccaatttatttgacgttttattttggttttcctcaccccttcaaactgtcggggagaccctgcccttcacctagctcccttggccaggcatgagggagccatggccttggtgaagctacctgcctcttctctcgcagccctgatgggggaaagggagtgggtactgcctgtggtttaggttcccctctccctttttctttttaattcaatttggaatcagaaagctgtggattctggcaaatggtcttgtgtcctttatcccactcaaacccatctggtcccctgttctccatagtccttcacccccaagcaccactgacagactggggaccagcccccttccctgcctgtgtctcttcccaaacccctctataggggtgacaagaagaggagggggggaggggacacgatccctcctcaggcatctgggaaggccttgcccccatgggctttaccctttcctgtgggctttctccctgacacatttgttaaaaatcaaacctgaataaaactacaagtttaatatgaaaaaaaaaaaaaaaaaaaaa

SEQ ID NO: 2 - protein, 154 aa, Rattus sp.
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 3 - DNA, 462 bp, Homo sapiens
atggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaa

SEQ ID NO: 4 - DNA, 460 bp, Homo sapiens
atggcagacgaaattgatttcactactggagatgccggggcttccagcacttaccctatgcagtgctcggccttgcgcaaaaacggcttcgtggtgctcaaaggacgaccatgcaaaatagtggagatgtcaacttccaaaactggaaagcatggtcatgccaaggttcaccttgttggaattgatattttcacgggcaaaaaatatgaagatatttgtccttctactcacaacatggatgttccaaatattaagagaaatgattatcaactgatatgcattcaagatggttacctttccctgctgacagaaactggtgaagttcgtgaggatcttaaactgccagaaggtgaactaggcaaagaaatagagggaaaatacaatgcaggtgaagatgtacaggtgtctgtcatgtgtgcaatgagtgaagaatatgctgtagccataaaaccctgcaaat

SEQ ID NO: 5 - DNA, 462 bp, Rattus sp.
atggcagatgatttggacttcgagacaggagatgcaggggcctcagccaccttcccaatgcagtgctcagcattacgtaagaatggttttgtggtgctcaaaggccggccatgtaagatcgtcgagatgtctacttcgaagactggcaagcatggccatgccaaggtccatctggttggcattgacatttttactgggaagaaatatgaagatatctgcccgtcgactcataatatggatgtccccaacatcaaacggaatgacttccagctgattggcatccaggatgggtacctatccctgctccaggacagtggggaggtacgagaggaccttcgtctgcctgaaggagaccttggcaaggagattgagcagaagtatgactgtggagaagagatcctgatcacagtgctgtctgccatgacagaggaggcagctgttgcaatcaaggccatggcaaaa

SEQ ID NO: 6 - DNA, 606 bp, Rattus sp.
  CDS (1)..(453)
gctgtgtattattgggcccataagaaccacatacctgtgctgagtcctgcactcacagacggctcactgggtgacatgatctttttccattcctataaaaacccaggcttggtcctggacatcgttgaagacctgcggctcatcaacatgcaggccattttcgccaagcgcactgggatgatcatcctgggtggaggcgtggtcaagcaccacatcgccaatgctaacctcatgcggaatggagctgactacgctgtttatatcaacacagcccaggagtttgatggctcagactcaggagcccggccagatgaggctgtctcctggggcaagatccggatggatgcacagccagtaaaggtctatgctgatgcatctctggttttccccttgctggtggctgagacattcgcccaaaaggcagatgccttcagagctgagaagaatgaggactgagcagatgggtaaagacggaggcttctgccacacctttatttattatttgcataccaacccctcctgggccctctccttggtcagcagcatcttgagaataaatggcctttttgttggtttctgtaaaaaaaggactttaaaaaaaaaaaa

SEQ ID NO: 7 - protein, 151 aa, Rattus sp.
AVYYWAHKNHIPVLSPALTDGSLGDMIFFHSYKNPGLVLDIVEDLRLINMQAIFAKRTGMIILGGGVVKHHIANANLMRNGADYAVYINTAQEFDGSDSGARPDEAVSWGKIRMDAQPVKVYADASLVFPLLVAETFAQKADAFRAEKNED

SEQ ID NO: 8 - DNA, 453 bp, Homo sapiens
tccgtgtattactgggcccagaagaaccacatccctgtgtttagtcccgcacttacagacggctcgctgggcgacatgatcttcttccattcctacaagaacccgggcctggtcctggacatcgttgaggacctgaggctcatcaacacacaggccatctttgccaagtgcactgggatgatcattctgggcgggggcgtggtcaagcaccacattgccaatgccaacctcatgcggaacggggccgactacgctgtttacatcaacacagcccaggagtttgatggctctgactcaggtgcccgaccagacgaggctgtctcctggggcaagatccgggtggatgcacagcccgtcaaggtctatgctgacgcctccctggtcttccccctgcttgtggctgaaacctttgcccagaagatggatgccttcatgcatgagaagaacgaggac

SEQ ID NO: 9 - DNA, 20 bp, Artificial Sequence
  Description of Artificial Sequence Synthetic Primer
  modified_base (12): a, t, c or g
tcsaarachggnaagcaygg

SEQ ID NO: 10 - DNA, 42 bp, Artificial Sequence
  Description of Artificial Sequence Synthetic Primer
gcgaagcttccatggctcgagttttttttttttttttttttt

SEQ ID NO: 11 - DNA, 972 bp, Rattus sp.
  CDS (1)..(327)
tcgaagaccggtaagcacggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaataactggcttccagggtggcggtggtggcagcagtgatccatgagcctacagaggcccctcccccagctctggctgggcccttggctggactcctatccaatttatttgacgttttattttggttttcctcaccccttcaaactgtcggggagaccctgcccttcacctagctcccttggccaggcatgagggagccatggccttggtgaagctacctgcctcttctctcgcagccctgatgggggaaagggagtgggtactgcctgtggtttaggttcccctctccctttttctttttaattcaatttggaatcagaaagctgtggattctggcaaatggtcttgtgtcctttatcccactcaaacccatctggtcccctgttctccatagtccttcacccccaagcaccactgacagactggggaccagcccccttccctgcctgtgtctcttcccaaacccctctataggggtgacaagaagaggagggggggaggggacacgatccctcctcaggcatctgggaaggccttgcccccatgggctttaccctttcctgtgggctttctccctgacacatttgttaaaaatcaaacctgaataaaactacaagtttaatatgaaaaaaaaaaaaaaaaaaaaa

SEQ ID NO: 12 - protein, 109 aa, Rattus sp.
SKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 13 - DNA, 24 bp, Artificial Sequence
  Description of Artificial Sequence Synthetic Primer
caggtctagagttggaatcgaagc

SEQ ID NO: 14 - DNA, 30 bp, Artificial Sequence
  Description of Artificial Sequence Synthetic Primer
atatctcgagccttgattgcaacagctgcc

SEQ ID NO: 15 - DNA, 489 bp, Rattus sp.
  CDS (33)..(485)
caggtctagagttggaatcgaagcctcttaaaatggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaagcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggctcgag

SEQ ID NO: 16 - protein, 151 aa, Rattus sp.
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKA

SEQ ID NO: 17 - DNA, 20 bp, Artificial Sequence
  Description of Artificial Sequence Synthetic Primer
gtctgtgtattattgggccc

SEQ ID NO: 18 - DNA, 42 bp, Artificial Sequence; identical to SEQ ID NO: 10
  Description of Artificial Sequence Synthetic Primer

SEQ ID NO: 19 - DNA, 1299 bp, Homo sapiens
ggcacgagggcggcggcggcggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtcttttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaa

SEQ ID NO: 20 - DNA, 462 bp, Rattus sp.
atggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaagcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaa

SEQ ID NO: 21 - protein, 154 aa, Homo sapiens; identical to SEQ ID NO: 2

SEQ ID NO: 22 - protein, 153 aa, Homo sapiens
MADEIDFTTGDAGASSTYPMQCSALRKNGFVVLKGRPCKIVEMSTSKTGKHGHAKVHLVGIDIFTGKKYEDICPSTHNMDVPNIKRNDYQLICIQDGYLSLLTETGEVREDLKLPEGELGKEIEGKYNAGEDVQVSVMCAMSEEYAVAIKPCK

SEQ ID NO: 23 - protein, 154 aa, Mus musculus; identical to SEQ ID NO: 2

SEQ ID NO: 24 - protein, 153 aa, Homo sapiens
MADEIDFTTGDAGASSTYPMQCSALRKNGFVVLKGRPCKIVEMSTSKTGKHGHAKVHLVGIDIFTGKKYEDICPSTHNMDVPNIKRNDYQLICIQDGCLSLLTETGEVREDLKLPEGELGKEIEGKYNAGEDVQVSVMCAMSEEYAVAIKPCK

SEQ ID NO: 25 - DNA, 20 bp, Artificial Sequence
  Description of Artificial Sequence Synthetic oligonucleotide
gacttggacttcgagacagg

SEQ ID NO: 26 - DNA, 19 bp, Artificial Sequence
  Description of Artificial Sequence Synthetic oligonucleotide
gcacggccacgccaaggtc

SEQ ID NO: 27 - DNA, 20 bp, Artificial Sequence
  Description of Artificial Sequence Synthetic oligonucleotide
ggacagcggggaggtacgag

SEQ ID NO: 28 - protein, 153 aa, Artificial Sequence; identical to SEQ ID NO: 24
  Description of Artificial Sequence Illustrative consensus sequence

SEQ ID NO: 29 - DNA, 1309 bp, Homo sapiens
ggcacgagggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtctttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaaaaaaaaaaaaaaaaaaaaaaaaa

SEQ ID NO: 30 - DNA, 23 bp, Homo sapiens
aaaggaatgacttccagctgatt

SEQ ID NO: 31 - DNA, 23 bp, Homo sapiens
aagatcgtcgagatgtctacttc

SEQ ID NO: 32 - DNA, 23 bp, Homo sapiens
aaggtccatctggttggtattga

SEQ ID NO: 33 - DNA, 23 bp, Homo sapiens
aagctggactcctcctacacaat

SEQ ID NO: 34 - DNA, 23 bp, Homo sapiens
aaagtcgaccttcagtaaggatt

SEQ ID NO: 35 - DNA, 20 bp, Homo sapiens
cctgtctcgaagtccaagtc

SEQ ID NO: 36 - DNA, 20 bp, Homo sapiens
gacttggacttcgagacagg

SEQ ID NO: 37 - DNA, 20 bp, Homo sapiens
ggaccttggcgtggccgtgc

SEQ ID NO: 38 - DNA, 20 bp, Homo sapiens
gcacggccacgccaaggtcc

SEQ ID NO: 39 - DNA, 20 bp, Homo sapiens
ctcgtacctccccgctctcc

SEQ ID NO: 40 - DNA, 19 bp, Homo sapiens
ggacagcggggaggtacga

SEQ ID NO: 41 - DNA, 465 bp, Homo sapiens
atggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataa

SEQ ID NO: 42 - DNA, 462 bp, Homo sapiens
atggcagacgaaattgatttcactactggagatgccggggcttccagcacttaccctatgcagtgctcggccttgcgcaaaaacggcttcgtggtgctcaaaggacgaccatgcaaaatagtggagatgtcaacttccaaaactggaaagcatggtcatgccaaggttcaccttgttggaattgatattttcacgggcaaaaaatatgaagatatttgtccttctactcacaacatggatgttccaaatattaagagaaatgattatcaactgatatgcattcaagatggttacctttccctgctgacagaaactggtgaagttcgtgaggatcttaaactgccagaaggtgaactaggcaaagaaatagagggaaaatacaatgcaggtgaagatgtacaggtgtctgtcatgtgtgcaatgagtgaagaatatgctgtagccataaaaccctgcaaataa

SEQ ID NO: 43 - protein, 154 aa, Homo sapiens [sequence omitted]

SEQ ID NO: 44 - DNA, 21 bp, Artificial Sequence
  Description of Artificial Sequence Synthetic oligonucleotide
aaaggaatgacttccagctga

SEQ ID NO: 45 - DNA, 23 bp, Artificial Sequence
  Description of Combined DNA/RNA Molecule Synthetic oligonucleotide
  Description of Artificial Sequence Synthetic oligonucleotide
aaaggaaugacuuccagcugatt

SEQ ID NO: 46 - DNA, 23 bp, Artificial Sequence
  Description of Combined DNA/RNA Molecule Synthetic oligonucleotide
  Description of Artificial Sequence Synthetic oligonucleotide
ucagcuggaagucauuccuuutt

SEQ ID NO: 47 - DNA, 21 bp, Artificial Sequence
  Description of Artificial Sequence Synthetic oligonucleotide
aagatcgtcgagatgtctact

SEQ ID NO: 48 - DNA, 23 bp, Artificial Sequence
  Description of Combined DNA/RNA Molecule Synthetic oligonucleotide
  Description of Artificial Sequence Synthetic oligonucleotide
aagaucgucgagaugucuacutt

SEQ ID NO: 49 - DNA, 23 bp, Artificial Sequence
  Description of Combined DNA/RNA Molecule Synthetic oligonucleotide
  Description of Artificial Sequence Synthetic oligonucleotide
aguagacaucucgacgaucuutt

SEQ ID NO: 50 - DNA, 21 bp, Artificial Sequence
  Description of Artificial Sequence Synthetic oligonucleotide
aaggtccatctggttggtatt

SEQ ID NO: 51 - DNA, 23 bp, Artificial Sequence
  Description of Combined DNA/RNA Molecule Synthetic oligonucleotide
  Description of Artificial Sequence Synthetic oligonucleotide
aagguccaucugguugguauutt

SEQ ID NO: 52 - DNA, 23 bp, Artificial Sequence
  Description of Combined DNA/RNA Molecule Synthetic oligonucleotide
  Description of Artificial Sequence Synthetic oligonucleotide
aauaccaaccagauggaccuutt

SEQ ID NO: 53 - DNA, 21 bp, Artificial Sequence
  Description of Artificial Sequence Synthetic oligonucleotide
aagctggactcctcctacaca

SEQ ID NO: 54 - DNA, 23 bp, Artificial Sequence
  Description of Combined DNA/RNA Molecule Synthetic oligonucleotide
  Description of Artificial Sequence Synthetic oligonucleotide
aagcuggacuccuccuacacatt

SEQ ID NO: 55 - DNA, 23 bp, Artificial Sequence
  Description of Combined DNA/RNA Molecule Synthetic oligonucleotide
  Description of Artificial Sequence Synthetic oligonucleotide
uguguaggaggaguccagcuutt

SEQ ID NO: 56 - DNA, 21 bp, Artificial Sequence
  Description of Artificial Sequence Synthetic oligonucleotide
aaagtcgaccttcagtaagga

SEQ ID NO: 57 - DNA, 23 bp, Artificial Sequence
  Description of Combined DNA/RNA Molecule Synthetic oligonucleotide
  Description of Artificial Sequence Synthetic oligonucleotide
aaagucgaccuucaguaaggatt

SEQ ID NO: 58 - DNA, 23 bp, Artificial Sequence
  Description of Combined DNA/RNA Molecule Synthetic oligonucleotide
  Description of Artificial Sequence Synthetic oligonucleotide
uccuuacugaaggucgacuuutt

SEQ ID NO: 59 - DNA, 26 bp, Artificial Sequence
  Description of Artificial Sequence Synthetic primer
gccaagcttaatggcagatgatttgg

SEQ ID NO: 60 - DNA, 25 bp, Artificial Sequence
  Description of Artificial Sequence Synthetic primer
ctgaattccagttattttgccatgg

SEQ ID NO: 61 - DNA, 27 bp, Artificial Sequence
  Description of Artificial Sequence Synthetic primer
aatgaattccgccatgacagaggaggc

SEQ ID NO: 62 - DNA, 42 bp, Artificial Sequence; identical to SEQ ID NO: 10
  Description of Artificial Sequence Synthetic primer

SEQ ID NO: 63 - DNA, 20 bp, Artificial Sequence
  Description of Artificial Sequence Synthetic oligonucleotide
cctgtctcgaagtccaagtc

SEQ ID NO: 64 - DNA, 20 bp, Artificial Sequence
  Description of Artificial Sequence Synthetic oligonucleotide
ggaccttggcgtggccgtgc

SEQ ID NO: 65 - DNA, 20 bp, Artificial Sequence
  Description of Artificial Sequence Synthetic oligonucleotide
ctcgtacctccccgctctcc

SEQ ID NO: 66 - DNA, 20 bp, Artificial Sequence
  Description of Artificial Sequence Synthetic oligonucleotide
cgtaccggtacggttccagg

SEQ ID NO: 67 - DNA, 20 bp, Artificial Sequence
  Description of Artificial Sequence Synthetic oligonucleotide
ggaccttggcgtggccgtgc

SEQ ID NO: 68 - protein, 17 aa, Artificial Sequence
  Description of Artificial Sequence Synthetic peptide
CRLPEGDLGKEIEQKYD

SEQ ID NO: 69 - DNA, 29 bp, Artificial Sequence
  Description of Artificial Sequence Synthetic oligonucleotide
aaaggaatgacttccagctgacctgtctc

SEQ ID NO: 70 - DNA, 29 bp, Artificial Sequence
  Description of Artificial Sequence Synthetic oligonucleotide
aatcagctggaagtcattcctcctgtctc

SEQ ID NO: 71 - DNA, 29 bp, Artificial Sequence
  Description of Artificial Sequence Synthetic oligonucleotide
aagatcgtcgagatgtctactcctgtctc

SEQ ID NO: 72 - DNA, 29 bp, Artificial Sequence
  Description of Artificial Sequence Synthetic oligonucleotide
aaagtagacatctcgacgatccctgtctc

SEQ ID NO: 73 - DNA, 29 bp, Artificial Sequence
  Description of Artificial Sequence Synthetic oligonucleotide
aaggtccatctggttggtattcctgtctc

SEQ ID NO: 74 - DNA, 29 bp, Artificial Sequence
  Description of Artificial Sequence Synthetic oligonucleotide
aaaataccaaccagatggacccctgtctc

SEQ ID NO: 75 - DNA, 29 bp, Artificial Sequence
  Description of Artificial Sequence Synthetic oligonucleotide
aagctggactcctcctacacacctgtctc

SEQ ID NO: 76 - DNA, 29 bp, Artificial Sequence
  Description of Artificial Sequence Synthetic oligonucleotide
aatgtgtaggaggagtccagccctgtctc

SEQ ID NO: 77 - DNA, 29 bp, Artificial Sequence
  Description of Artificial Sequence Synthetic oligonucleotide
aaagtcgaccttcagtaaggacctgtctc

SEQ ID NO: 78 - DNA, 29 bp, Artificial Sequence
  Description of Artificial Sequence Synthetic oligonucleotide
aatccttactgaaggtcgactcctgtctc

SEQ ID NO: 79 - RNA, 20 nt, Artificial Sequence
  Description of Artificial Sequence Synthetic oligonucleotide
aagcuggacuccuccuacac

SEQ ID NO: 80 - RNA, 21 nt, Artificial Sequence
  Description of Artificial Sequence Synthetic oligonucleotide
aaacacauccuccucaggucg

SEQ ID NO: 81 - DNA, 21 bp, Artificial Sequence
  Description of Artificial Sequence Synthetic oligonucleotide
aaaggaatgacttccagctga

SEQ ID NO: 82 - DNA, 21 bp, Artificial Sequence
  Description of Artificial Sequence Synthetic oligonucleotide
aagatcgtcgagatgtctact

SEQ ID NO: 83 - DNA, 21 bp, Artificial Sequence
  Description of Artificial Sequence Synthetic oligonucleotide
aaggtccatctggttggtatt

SEQ ID NO: 84 - DNA, 21 bp, Artificial Sequence
  Description of Artificial Sequence Synthetic oligonucleotide
aagctggactcctcctacaca

SEQ ID NO: 85 - DNA, 21 bp, Artificial Sequence
  Description of Artificial Sequence Synthetic oligonucleotide
aaagtcgaccttcagtaagga

SEQ ID NO: 86 - RNA, 21 nt, Artificial Sequence
  Description of Combined DNA/RNA Molecule Synthetic oligonucleotide
  Description of Artificial Sequence Synthetic siRNA sequence
  misc_feature (1)..(2): a, t, c, g or u
nngcuggacuccuccuacaca

SEQ ID NO: 87 - DNA, 21 bp, Artificial Sequence
  Description of Combined DNA/RNA Molecule Synthetic oligonucleotide
  Description of Artificial Sequence Synthetic siRNA sequence
gcuggacuccuccuacacatt

SEQ ID NO: 88 - DNA, 21 bp, Artificial Sequence
  Description of Artificial Sequence Synthetic siRNA sequence
uguguaggaggaguccagctt

SEQ ID NO: 89 - RNA, 21 nt, Artificial Sequence
  Description of Combined DNA/RNA Molecule Synthetic oligonucleotide
  Description of Artificial Sequence Synthetic siRNA sequence
  misc_feature (1)..(2): a, t, c, g or u
nnacacauccuccucaggucg

SEQ ID NO: 90 - DNA, 21 bp, Artificial Sequence
  Description of Combined DNA/RNA Molecule Synthetic oligonucleotide
  Description of Artificial Sequence Synthetic siRNA sequence
acacauccuccucaggucgtt

SEQ ID NO: 91 - DNA, 21 bp, Artificial Sequence
  Description of Combined DNA/RNA Molecule Synthetic oligonucleotide
  Description of Artificial Sequence Synthetic siRNA sequence
cgaccugaggaggaugugutt

SEQ ID NO: 92 - DNA, 24 bp, Artificial Sequence
  Description of Artificial Sequence Synthetic siRNA sequence
cggatggcaacatttagaattagt

SEQ ID NO: 93 - DNA, 24 bp, Artificial Sequence
  Description of Artificial Sequence Primer
tgattgagactgtaatcaagaacc

SEQ ID NO: 94 - DNA, 22 bp, Artificial Sequence
  Description of Artificial Sequence Primer
ctgatgcccccatgttcgtcat

SEQ ID NO: 95 - DNA, 20 bp, Artificial Sequence
  Description of Artificial Sequence Primer
ccaccaccctgttgctgtag

(19) United States
(12) Patent Application Publication
(10) Pub. No.: US 2007/0154457 A1
(43) Pub. Date: Jul. 5, 2007

(54) Use of eIF-5A to kill multiple myeloma cells

(75) Inventors: John E. Thompson, Waterloo (CA); Catherine A. Taylor, Waterloo (CA)

Correspondence Address: KENYON & KENYON LLP, 1500 K STREET N.W., SUITE 700, WASHINGTON, DC, 20005, US

(73) Assignee: Senesco Technologies, Inc., New Brunswick, NJ (US)

(21) Appl. No.: 11/637,835

(22) Filed: Dec. 13, 2006

Related U.S. Application Data
(60) Provisional application No. 60/749,604, filed on Dec. 13, 2005.
(60) Provisional application No. 60/795,168, filed on Apr. 27, 2006.

(51) Int. Cl. A61K 48/00; A61K 9/127; A61K 31/787

(52) U.S. Cl. 424/93.2; 514/44; 424/450; 424/78.3; 977/906; 977/754

(57) ABSTRACT

The present invention relates to eucaryotic initiation factor 5A and the use of polynucleotides encoding the same to inhibit cancer cell growth and inhibit metastases. In a preferred embodiment, eIF-5A1 is used to kill multiple myeloma cells.

### RELATED APPLICATIONS

This application claims priority to U.S. provisional application 60/749,604, filed on Dec. 13, 2005 and 60/795,168, filed on Apr. 27, 2006, both of which are incorporated by reference in their entirety.

### FIELD OF THE INVENTION

The present invention relates to apoptosis-specific eukaryotic initiation factor (“eIF-5A”) and the use of polynucleotides encoding the same to kill multiple myeloma cells, as well as other cancer cells. The present invention relates to the use of apoptosis-specific eIF-5A or referred to as “apoptosis-specific eIF-5A” or “eIF-5A1” as well as the use of the eIF-5A2 isoform to inhibit multiple myeloma, kill multiple myeloma cells, and to inhibit and/or kill other cancer cell growth.

### BACKGROUND OF THE INVENTION

Apoptosis is a genetically programmed cellular event that is characterized by well-defined morphological features, such as cell shrinkage, chromatin condensation, nuclear fragmentation, and membrane blebbing. Kerr et al. (1972) Br. J. Cancer, 26, 239-257; Wyllie et al. (1980) Int. Rev. Cytol., 68, 251-306. It plays an important role in normal tissue development and homeostasis, and defects in the apoptotic program are thought to contribute to a wide range of human disorders ranging from neurodegenerative and autoimmunity disorders to neoplasms. Thompson (1995) Science, 267, 1456-1462; Mullauer et al. (2001) Mutat. Res, 488, 211-231. Although the morphological characteristics of apoptotic cells are well characterized, the molecular pathways that regulate this process have only begun to be elucidated.

Another key protein involved in apoptosis is a protein that encoded by the tumor suppressor gene p53. This protein is a transcription factor that regulates cell growth and induces apoptosis in cells that are damaged and genetically unstable, presumably through up-regulation of Bax. Bold et al. (1997) Surgical Oncology, 6, 133-142; Ronen et al., 1996; Schuler & Green (2001) Biochem. Soc. Trans., 29, 684-688; Ryan et al. (2001) Curr. Opin. Cell Biol., 13, 332-337; Zornig et al. (2001) Biochem. Biophys. Acta, 1551, F1-F37.

Alterations in the apoptotic pathways are believed to play a key role in a number of disease processes, including cancer. Wyllie et al. (1980) Int. Rev. Cytol., 68, 251-306; Thompson (1995) Science, 267, 1456-1462; Sen & D'Incalci (1992) FEBS Letters, 307, 122-127; McDonnell et al. (1995) Seminars in Cancer and Biology, 6, 53-60. Investigations into cancer development and progression have traditionally been focused on cellular proliferation. However, the important role that apoptosis plays in tumorigenesis has recently become apparent. In fact, much of what is now known about apoptosis has been learned using tumor models, since the control of apoptosis is invariably altered in some way in tumor cells. Bold et al. (1997) Surgical Oncology, 6, 133-142.

Cytokines also have been implicated in the apoptotic pathway. Biological systems require cellular interactions for their regulation, and cross-talk between cells generally involves a large variety of cytokines. Cytokines are mediators that are produced in response to a wide variety of stimuli by many different cell types. Cytokines are pleiotropic molecules that can exert many different effects on many different cell types, but are especially important in regulation of the immune response and hematopoietic cell proliferation and differentiation. The actions of cytokines on target cells can promote cell survival, proliferation, activation, differentiation, or apoptosis depending on the particular cytokine, relative concentration, and presence of other mediators.

Deoxyhypusine synthase (DHS) and hypusine-containing eukaryotic translation initiation Factor-5A (eIF-5A) are known to play important roles in many cellular processes including cell growth and differentiation. Hypusine, a unique amino acid, is found in all examined eukaryotes and archaebacteria, but not in eubacteria, and eIF-5A is the only known hypusine-containing protein. Park (1988) J. Biol. Chem., 263, 7447-7449; Schumann & Klink (1989) System. Appl. Microbiol., 11, 103-107; Bartig et al. (1990) System. Appl. Microbiol., 13, 112-116; Gordon et al. (1987a) J. Biol. Chem., 262, 16585-16589. Active eIF-5A is formed in two post-translational steps: the first step is the formation of a deoxyhypusine residue by the transfer of the 4-aminobutyl moiety of spermidine to the α-amino group of a specific lysine of the precursor eIF-5A catalyzed by deoxyhypusine synthase; the second step involves the hydroxylation of this 4-aminobutyl moiety by deoxyhypusine hydroxylase to form hypusine.

The amino acid sequence of eIF-5A is well conserved between species, and there is strict conservation of the amino acid sequence surrounding the hypusine residue in eIF-5A, which suggests that this modification may be important for survival. Park et al. (1993) Biofactors, 4, 95-104. This assumption is further supported by the observation that inactivation of both isoforms of eIF-5A found to date in yeast, or inactivation of the DHS gene, which catalyzes the first step in their activation, blocks cell division. Schnier et al. (1991) Mol. Cell. Biol., 11, 3105-3114; Sasaki et al. (1996) FEBS Lett., 384, 151-154; Park et al. (1998) J. Biol. Chem., 273, 1677-1683. However, depletion of eIF-5A protein in yeast resulted in only a small decrease in total protein synthesis suggesting that eIF-5A may be required for the translation of specific subsets of mRNA's rather than for protein global synthesis. Kang et al. (1993), “Effect of initiation factor eIF-5A depletion on cell proliferation and protein synthesis,” in Tuite, M. (ed.), Protein Synthesis and Targeting in Yeast, NATO Series H. The recent finding that ligands that bind eIF-5A share highly conserved motifs also supports the importance of eIF-5A. Xu & Chen (2001) J. Biol. Chem., 276, 2555-2561. In addition, the hypusine residue of modified eIF-5A was found to be essential for sequence-specific binding to RNA, and binding did not provide protection from ribonucleases.

In addition, intracellular depletion of eIF-5A results in a significant accumulation of specific mRNAs in the nucleus, indicating that eIF-5A may be responsible for shuttling specific classes of mRNAs from the nucleus to the cytoplasm. Liu & Tartakoff (1997) Supplement to Molecular Biology of the Cell, 8, 426a. Abstract No. 2476, 37th American Society for Cell Biology Annual Meeting. The accumulation of eIF-5A at nuclear pore-associated intranuclear filaments and its interaction with a general nuclear export receptor further suggest that eIF-5A is a nucleocytoplasmic shuttle protein, rather than a component of polysomes. Rosorius et al. (1999) J. Cell Science, 112, 2369-2380.

The first cDNA for eIF-5A was cloned from human in 1989 by Smit-McBride et al., and since then cDNAs or genes for eIF-5A have been cloned from various eukaryotes including yeast, rat, chick embryo, alfalfa, and tomato. Smit-McBride et al. (1989) J. Biol. Chem., 264, 1578-1583; Schnier et al. (1991) (yeast); Sano, A. (1995) in Imahori, M. et al. (eds), Polyamines, Basic and Clinical Aspects, VNU Science Press, The Netherlands, 81-88 (rat); Rinaudo & Park (1992) FASEB J., 6, A453 (chick embryo); Pay et al. (1991) Plant Mol. Biol., 17, 927-929 (alfalfa); Wang et al. (2001) J. Biol. Chem., 276, 17541-17549 (tomato).

Multiple myeloma is a progressive and fatal disease characterized by the expansion of malignant plasma ells in the bone marrow and by the presence of osteolytic lesions. Multiple myeloma is an incurable but treatable cancer of the plasma cell. Plasma cells are an important part of the immune system, producing immunoglobulins (antibodies) that help fight infection and disease. Multiple myeloma is characterized by excessive numbers of abnormal plasma cells in the bone marrow and overproduction of intact monoclonal immunoglobulins (IgG, IgA, IgD, or IgE; “M-proteins”) or Bence-Jones protein (free monoclonal light chains). Hypocalcaemia, anemia, renal damage, increased susceptibility to bacterial infection, and impaired production of normal immunoglobulin are common clinical manifestations of multiple myeloma. Multiple myeloma is often also characterized by diffuse osteoporosis, usually in the pelvis, spine, ribs, and skull.

Conventional therapies for of multiple myeloma include chemotherapy, stem cell transplantation, high-dose chemotherapy with stem cell transplantation, and salvage therapy. Chemotherapies include treatment with Thalomid®(thalidomide), bortezomib, Aredia® (pamidronate), steroids, and Zometa® (zoledronic acid). However many chemotherapy drugs are toxic to actively dividing non-cancerous cells, such as of the bone marrow, the lining of the stomach and intestines, and the hair follicles. Therefore, chemotherapy may result in a decrease in blood cell counts, nausea, vomiting, diarrhea, and loss of hair.

Conventional chemotherapy, or standard-dose chemotherapy, is typically the primary or initial treatment for patients with of multiple myeloma. Patients also may receive chemotherapy in preparation for high-dose chemotherapy and stem cell transplant. Induction therapy (conventional chemotherapy prior to a stem cell transplant) can be used to reduce the tumor burden prior to transplant. Certain chemotherapy drugs are more suitable for induction therapy than others, because they are less toxic to bone marrow cells and result in a greater yield of stem cells from the bone marrow. Examples of chemotherapy drugs suitable for induction therapy include dexamethasone, thalidomide/dexamethasone, VAD (vincristine, Adriamycin® (doxorubicin), and dexamethasone in combination), and DVd (pegylated liposomal doxorubicin (Doxil®, Caelyx®), vincristine, and reduced schedule dexamethasone in combination).

The standard treatment for of multiple myeloma is melphalan in combination with prednisone (a corticosteroid drug), achieving a response rate of 50%. Unfortunately, melphalan is an alkylating agent and is less suitable for induction therapy. Corticosteroids (especially dexamethasone) are sometimes used alone for multiple myeloma therapy, especially in older patients and those who cannot tolerate chemotherapy. Dexamethasone is also used in induction therapy, alone or in combination with other agents. VAD is the most commonly used induction therapy, but DVd has recently been shown to be effective in induction therapy. Bortezomib has been approved recently for the treatment of multiple myeloma, but it is very toxic. However, none of the existing therapies offer a significant potential for a cure. Thus, there remains a need for a suitable therapy to kill multiple myeloma cells. The present invention provides this need.

### SUMMARY OF INVENTION

The present invention provides a method of inhibiting cancer cell growth and/or killing cancer cells. The present invention also provides a method of inhibiting or slowing down the ability of a cancer cell to metastasize. Inhibiting cancer growth includes a reduction in the size of a tumor, a decrease in the growth of the tumor, and can also encompass a complete remission of the tumor. The cancer can be any cancer or tumor, including but not limited to colon cancer, colorectal adenocarcinoma, bladder carcinoma, cervical adenocarcinoma, and lung carcinoma. The methods of the present invention involve the administration of eIF-5A, preferably human eIF-5A1 to a patient (a mammal, preferably a human) having said cancer. The eIF-5A2 isoform may also be used, although eIF-5A1 is preferred. The eIF-5A may be delivered to a subject in need thereof by any suitable method know in the art. It may be delivered as naked DNA, such as DNA in biologically suitable medium and delivered through IV or subcutaneous injection or any other biologically suitable delivery mechanism. Alternatively, the eIF-5A may be delivered in a vector such as an adenovirus vector. Alternatively, the DNA may be delivered in liposomes or any other suitable “carrier” that provided for delivery of the DNA to the target cancer cells. The

eIF-5A may also be delivered directly to the site of the tumor. One skilled in the art would be able to determine the dose and length of treatment regimen for delivery of eIF-5A. eIF-5A1 and eIF-5A2 is known and has been described in earlier co-pending applications, such as Ser. Nos. 09/909,796 (U.S. Pat. No. 6,867,237); 10/141,647 (allowed); 10/200,148; 10/277,969; 10/383,614; 10/792,893; 11/287,460; 10/861,980; 11/134,445; 11/184,982; 11/293,391; 60/749,604; and 60/795,168, which are all herein incorporated by reference. Since eIF-5As are highly conserved among species, any eIF-5A may be used in the present invention, human, rat, mouse, dog etc. Preferably, a human eIF-5A would be used for treatment of humans, etc. The eIF-5A also includes mutant eIF-5As, as long as the mutant is capable of up-regulating or increasing expression of eIF-5A and hence inhibit the growth of cancer or kill cancer cells.

The present invention also provides for a method of activating MAPK/SAPK signaling pathway in a cell by providing a nucleotide encoding eIF-5A1 to said cells. The eIF-5A1 polynucleotide and eIF-5A1 protein is as described above.

The present invention also provides pharmaceutical compositions useful for killing myeloma cells comprising polynucleotides encoding eIF5A. The eIF5A maybe eIF5A1, eIF5A2 or a mutant eIF5A1. Preferably the eIF5A is eIF5A1. The composition may further comprise a delivery vehicle. The delivery vehicle may be, but is not limited to, a vector, plasmid, liposome, or dendrimer.

The present invention also provides the use of eIF5A (preferably eIF5A1) to make a medicament to kill multiple myeloma cells in a subject having multiple myeloma.

The present invention further provides a method of killing multiple myeloma cells comprising administering to the myeloma cells a composition comprising a polynucleotide encoding eIF5A1, wherein the composition kills the multiple myeloma cells. The eIF5A1 may be a mutant, wherein the mutant has had the conserved lysine changed to another amino acid and wherein said mutant is unable to be hypusinated. Compositions useful in the methods of treatment are as described herein.

The present invention further provides a method of killing multiple myeloma cells wherein a composition comprising siRNA directed against eIF-5A1 is provided in addition to a composition comprising polynucleotides encoding eIF5A1. The siRNA down regulates endogenous expression of eIF-5A1, and thus down regulates expression of IL-6, which in turn causes in apoptosis in myeloma cells. The composition comprising the eIF-5A1 siRNA may be administered intravenously or administered within a delivery vehicle such as a plasmid, vector, liposome or dendrimer.

### BRIEF DESCRIPTION OF THE FIGURES

FIG. 1 shows that eIF5A1 expression is increased by genotoxic stress and nitric oxide. A) Northern blot (top panel) and Western blot (bottom panel) analysis of eIF5A expression in normal colon fibroblasts treated with 0.5 μg/ml Actinomycin D for 0, 1, 4, 8, and 24 hours. The Western blot was probed with antibodies against eIF5A, p53, and β-actin. B) Northern blot (top panel) and Western blot (bottom panel) analysis of eIF5A expression in RKO cells treated with 3 mM sodium nitroprusside for 0, 2, 4, 8, and 24 hours. The Western blot was probed with antibodies against eIF5A and β-actin. The RKO cells do not express eIF5A2 at significant levels. Further, eIF-5A2 is only one amino acid longer in size than eIF-5A1, but it runs higher on an SDS PAGE gel, so that if eIF5A2 were expressed, it would be seen as a separate band from eIF-5A1.

FIG. 2 shows suppression of eIF5A1 expression has no effect on cell proliferation. A) Western blots of cell lysates isolated from HT-29 cells 72 hours after transfection with either eIF5A1 siRNA or control siRNA. Western blots from two independent experiments are shown. The blots were probed with antibodies against eIF5A and β-actin. B) The metabolic activity of cells transfected with eIF5A1 siRNA was measured using an XTT cell proliferation assay. HT-29 cells were seeded on a 96-well plate 24 hours before transfection with either control siRNA or eIF5A1 siRNA. Twenty-four hours after transfection, the cells were either left untreated or treated with Actinomycin D (1.0 μg/ml) for 48 hours before measuring metabolic activity. Values are means for two experiments performed in quadruplicate and were normalized to the value obtained for the 0 hour control which was set at 1. C) The proliferative ability of HT-29 cells transfected with control or eIF5A1 siRNA was compared to that of cells incubated with 50 μM GC7 for 72 hours. Cell proliferation was measured by BrdU incorporation. Values are means±SE for n=4 and were normalized to the value for the GC7 (+) serum sample which was set at 1. Asterisks (*) denote values considered significantly different by paired Student t-test (p<0.01). D) XTT and E) BrdU incorporation cell proliferation assays of HT-29 cells transfected with either control siRNA or eIF5A siRNA from the day of transfection (day 0) through to day 5 after transfection. Values for day 0 were set at 1.

FIG. 3 shows that eIF5A1 regulates expression of p53 in response to Actinomycin D. RKO cells were transfected with either control siRNA (C) or eIF5A siRNA (5A-1). Seventy-two hours after transfection, the cells were treated for 0, 4, 8, or 24 hours with 0.5 μg/ml Actinomycin D. A) Western blot of cell lysates blotted with antibodies against eIF5A, p53, or actin. The result is representative of three independent experiments. B) Plot of the relative intensities of p53 in Western blots that were normalized to the corresponding actin bands. Values are means±SE for a minimum of n=3.

FIG. 4 shows that overexpression of human eIF5A1 induces apoptosis independently of p53. RKO cells (A) or RKO-E6 cells (B) were transfected with pHM6-LacZ, pHM6 eIF5A, or pHM6-eIF5AA37 (a 37 amino acid truncation of the C-terminus). Forty-eight hours after transfection, the cells were fixed and labeled using the TUNEL method. The nuclei were stained with Hoescht 33258, and the labeled cells were viewed by fluorescence microscopy. Cells stained bright green were scored as apoptotic. Hoescht-stained nuclei were used to determine the total cell number. Values are means±SE for n=4 (A) or n=3 (B). Asterisks (*) denote significant difference from the control (pHM6-LacZ) by paired Student t-test (p<0.02). C) Western blot of RKO and RKO-E6 cell lysates harvested 0, 4, 8 and 24 hours after treatment with 0.5 μg/ml Actinomycin D. Blots were probed with antibodies against p53 and β-actin.

FIG. 5 shows that eIF5A1 adenovirus constructs induce apoptosis in colon cancer cells. A) Western blot of cell lysate isolated from HT-29 cells seventy-two hours after infection with Ad-LacZ (L), AdeIF5A1 (5A), or Ad-eIF5A(K50A) (K50A). B) 2-D gel electrophoresis of cell lysate isolated from HT-29 cells after treatment with GC7 or DFO for seventy-two hours or seventy-two hours after infection with adenovirus constructs followed by Western blotting with an antibody against eIF5A. 7 μg of protein was separated except for lysate from Adenovirus-infected cells overexpressing eIF5A for which 0.3 μg of protein was separated. C) Top panel: TUNEL staining of HT-29 cells forty-eight hours after infection with adenovirus constructs. Bottom panel: Hoechst-stained nuclei of cells in the same field. All photographs were taken at 400× magnification. The results are representative of three independent experiments. D) Percent apoptosis of HT-29 cells forty-eight hours after infection with adenovirus constructs. Values are means±SE for n=3. E) XTT cell proliferation assay of HT-29 cells seven days after infection with adenovirus constructs. Values are means±SE for n=4.

FIG. 6 shows Annexin V and PI staining of HT-29 cells infected with eIFA1 adenovirus constructs. A) Histograms of Annexin-FITC and propidium iodide (PI) labeling of HT-29 cells forty-eight after infection with adenovirus constructs. B) Percent apoptosis of HT-29 cells, as determined by Annexin V labeling and flow cytometry analysis, twenty-four, forty-eight, and seventy-two hours after infection with adenovirus constructs or after treatment with the apoptosis-inducing agents, Actinomycin D or Brefeldin A. Values are means±SE for n=2 (# of events >5000).

FIG. 7 shows immunofluorescent localization of eIF5A1. The subcellular localization of eIF5A protein in HT-29 cells stimulated with IFN-γ and TNF-α (A) or Actinomycin D (B) was determined by indirect immunofluorescence. Hoechst 33258 was used to stain the nuclei. A) HT-29 cells were either untreated or primed with IFN-γ for 16 hours before stimulation with TNF-α for 0 min. [(−) TNF-α], 10 min. or 30 min. Top panel: immunofluorescent detection of eIF5A1; middle panel: Hoechst-stained nuclei of cells in the same field; bottom panel: merged images. B) HT-29 cells were either untreated or treated with Actinomycin D for 0.5 hr, 1.5 hrs. or 4 hrs. Top panel: immunofluorescent detection of eIF5A1; middle panel: Hoechst-stained nuclei of cells in the same field; bottom panel: merged images. All photographs were taken at 400× magnification. The results are representative of three independent experiments.

FIG. 8 shows that eIF5A1 regulates expression of p53 in response to Actinomycin D. RKO-E6 cells do not express p53. A) RKO or RKO-E6 cells were treated with 0.5 μg/ml Actinomycin D for 1, 4, 8, or 24 hours. Cell lysate was harvested and analyzed for p53 expression using Western blotting. B) RKO cells were transfected with either control siRNA (C), eIF5A siRNA (5A-1) or a second eIF5A siRNA targeting a different region of eIF5A (5A-2). Seventy-two hours after transfection, the cells were treated for 0, 4, 8, or 24 hours with 0.5 μg/ml Actinomycin D. A) Western blot of cell lysates blotted with antibodies against eIF5A, p53, or actin. The result is representative of three independent experiments.

FIG. 9 shows that deoxyhypusinated eIF5A1 accumulates during apoptosis. This figure provides a 2-D gel electrophoresis of cell lysate isolated from HT-29 cells after treatment with Actinomycin D (A) or an agonist Fas antibody (B) for times ranging from 1 to 24 hours followed by Western blotting with an antibody against eIF5A.

FIG. 10 shows that infection with Ad-eIF5A1 or Ad-eIF5A1(K50A) {Ad-eIF5A1M) induces apoptosis in HT-29 colorectal adenocarcinoma cells. This figure provides the percent apoptosis of HT-29 cells, as determined by Annexin V labeling and flow cytometry analysis, twenty-four, forty-eight, and seventy-two hours after infection with adenovirus constructs or after treatment with the apoptosis-inducing agents, Actinomycin D or Brefeldin A. Values are means±SE for n=2 (# of events >5000).

FIG. 11 shows that infection with Ad-eIF5A1 or Ad-eIF5A2 induces apoptosis in HTB-9 bladder carcinoma cells. This figure provides percent apoptosis of HTB-9 cells, as determined by Annexin V labeling and flow cytometry analysis, twenty-four, forty-eight, and seventy-two hours after infection with adenovirus constructs or after treatment with the apoptosis-inducing agents, Actinomycin D or Brefeldin A. Values are means±SE for n=2 (# of events >5000).

FIG. 12 shows that infection with Ad-eIF5A1 or Ad-eIF5A2 induces apoptosis in HTB-4 bladder carcinoma cells. This figure provides percent apoptosis of HTB-4 cells, as determined by Annexin V labeling and flow cytometry analysis, twenty-four, forty-eight, and seventy-two hours after infection with adenovirus constructs or after treatment with the apoptosis-inducing agents, Actinomycin D or Brefeldin A. Values are means±SE for n=2 (# of events >5000).

FIG. 13 shows that infection with Ad-eIF5A1, Ad-eIF5A1(K50A) {Ad-eIF5A1M}, or Ad-eIF5A2 inhibits growth of HTB-4 bladder carcinoma cells. This figure provides the results of an XTT cell proliferation assay of HTB-4 cells twenty-four, forty-eight, and seventy-two after infection with adenovirus constructs. Values are means±SE for n=2.

FIG. 14 shows that infection with Ad-eIF5A1, Ad-eIF5A1(K50A) {Ad-eIF5A1M}, or Ad-eIF5A2 inhibits growth of HTB-9 bladder carcinoma cells. This figure provides the results of an XTT cell proliferation assay of HTB-9 cells twenty-four, forty-eight, and seventy-two after infection with adenovirus constructs. Values are means±SE for n=2.

FIG. 15 shows that infection with Ad-eIF5A1, Ad-eIF5A1(K50A) {Ad-eIF5A1M}, or Ad-eIF5A2 inhibits growth of HTB-1 bladder carcinoma cells. This figure provides the results of an XTT cell proliferation assay of HTB-1 cells twenty-four, forty-eight, and seventy-two after infection with adenovirus constructs. Values are means±SE for n=2.

FIG. 16 shows that infection with Ad-eIF5A1, Ad-eIF5A1(K50A) {Ad-eIF5A1M}, or Ad-eIF5A2 inhibits growth of UMUC-3 bladder carcinoma cells. This figure provides the results of an XTT cell proliferation assay of UMUC-3 cells twenty-four, forty-eight, and seventy-two after infection with adenovirus constructs. Values are means±SE for n=2.

FIG. 17 shows that infection with Ad-eIF5A1, Ad-eIF5A1(K50A) {Ad-eIF5A1M}, or Ad-eIF5A2 inhibits growth of HeLa cervical adenocarcinoma cells. This figure provides the results of an XTT cell proliferation assay of HeLa cells twenty-four, forty-eight, and seventy-two after infection with adenovirus constructs. Values are means±SE for n=2.

FIG. 18 shows that infection with Ad-eIF5A1, Ad-eIF5A1(K50A) (Ad-eIF5A1M), or Ad-eIF5A2 induces PARP cleavage in HTB-4 cells. This figure provides the results of a Western blot of cell lysate isolated from HTB-4 cells forty-eight or seventy-two hours after infection with adenovirus constructs using PARP, eIF5A and β-actin antibodies.

FIG. 19 shows that infection with Ad-eIF5A1, Ad-eIF5A1(K50A) (Ad-eIF5A1M), or Ad-eIF5A2 does not induce PARP cleavage in HTB-1 cells. This figure provides the results of a Western blot of cell lysate isolated from HTB-1 cells forty-eight or seventy-two hours after infection with adenovirus constructs using PARP, eIF5A and β-actin antibodies.

FIG. 20 shows that over-expression of eIF5A1 in A549 lung carcinoma cells activates MAPK/SAPK signaling pathways. A549 cells were infected with Ad-eIF5A1 (5A) at increasing multiplicities of infection (MOI). Cells were infected with Ad-LacZ (Lac) for comparison. Forty-eight hours after infection, the cells were treated with EGF for 30 minutes and the cell lysate was harvested for Western blot analysis.

FIG. 21 shows that over-expression of eIF5A1 in A549 lung carcinoma cells activates MAPK/SAPK signaling pathways. A549 cells were infected with Ad-eIF5A1 (5A) or with Ad-LacZ (L) at 50 MOI. Cells were treated with EGF for 30 minutes either 6, 24, 48, or 72 hours after infection and the cell lysate was harvested for Western blot analysis.

FIG. 22 shows that over-expression of a mutant eIF5A1 that is incapable of being hypusinated activates MAPK/SAPK signaling pathways. A549 cells were infected with Ad-eIF5A1(K50A) (M) at increasing multiplicities of infection (MOI). Cells were infected with Ad-eIF5A1 (5A) and Ad-LacZ (Lac) for comparison. Cells were incubated with or without the MEK inhibitor U1026. Forty-eight hours after infection, the cells were treated with EGF for 30 minutes and the cell lysate was harvested for Western blot analysis.

FIG. 23 shows that over-expression of eIF5A 1 or mutant eIF5A 1 (K50A) in A549 lung carcinoma cells increases expression of p53. A549 cells were infected with Ad-eIF5A1 (5A) or Ad-eIF5A1(K50A) (M) at increasing multiplicities of infection (MOI). Cells were infected with Ad-LacZ (Lac) for comparison. Cells were incubated with or without the MEK inhibitor U1026. Forty-eight hours after infection, the cells were treated with EGF for 30 minutes and the cell lysate was harvested for Western blot analysis.

FIG. 24 shows that over-expression of eIF5A1 or mutant eIF5A1(K50A) reduces the capacity of A549 lung carcinoma cells to invade through Matrigel™ extracellular matrix. Twenty-four hours after infection with Adenovirus constructs, A549 cells were seeded onto Matrigel™-coated transwells in serum-free media. Serum-containing media was placed in the bottom well to act as a chemoattractant. Twenty-four hours after plating, cells which had invaded through to the bottom surface of the transwell were stained with crystal violet and the average number of cells per field was determined by counting cells under a light microscope. A minimum of six fields per sample were counted.

FIG. 25 shows the results of experiment II: Lung tumor load was significantly reduced by eIF5A1 in B16F10-bearing C57BL/6 mice. 200,000 B16F10 cells were injected into the tail vein of 6-week old C57BL/6 mice. Plasmid DNA bearing either the LacZ gene (as a DNA control) or eIF5A1 was injected into the tail vein on days 2, 4, 7, 11, 16, 21, 26, and 31. Three different concentrations of DNA were used: 1× (3.3 mg/kg), 0.1× (0.3 mg/kg), and 2× (6.6 mg/kg). Mice which received PBS instead of B16F10 cells were used as a negative control while B16F10-bearing mice that received injections of PBS rather than plasmid DNA were used as a positive control. When the mice became moribund, they were sacrificed and the lungs were removed and photographed

FIG. 26 shows the result of experiment II: Lung weight was significantly reduced by eIF5A1 in B16F10-bearing C57BL/6 mice. 200,000 B16F10 cells were injected into the tail vein of 6-week old C57BL/6 mice. Plasmid DNA bearing either the LacZ gene (as a DNA control) or eIF5A1 was injected into the tail vein on days 2, 4, 7, 11, 16, 21, 26, and 31. Three different concentrations of DNA were used: 1× (3.3 mg/kg), 0.1× (0.3 mg/kg), and 2× (6.6 mg/kg). Mice that received PBS instead of B16F10 cells were used as a negative control while B16F10-bearing mice that received injections of PBS rather than plasmid DNA were used as a positive control. When the mice became moribund, they were sacrificed and the lungs were removed and weighed.

FIG. 27 shows the results of experiment II: VEGF expression was significantly reduced by eIF5A1 in B16F10-bearing C57BL/6 mice. 200,000 B16F10 cells were injected into the tail vein of 6-week old C57BL/6 mice. Plasmid DNA bearing either the LacZ gene (as a DNA control) or eIF5A1 was injected into the tail vein on days 2, 4, 7, 11, 16, 21, 26, and 31. Three different concentrations of DNA were used: 1× (3.3 mg/kg), 0.1× (0.3 mg/kg), and 2× (6.6 mg/kg). Mice that received PBS instead of B16F10 cells were used as a negative control while B16F10-bearing mice that received injections of PBS rather than plasmid DNA were used as a positive control. When the mice became moribund, they were sacrificed and the lungs were removed and weighed. Once lung weight was determined, the lungs were frozen and later used for VEGF ELISAs. Lung tissue was ground in lysis buffer and the amount of VEGF present in the lysate was determined by ELISA.

FIG. 28 shows the results of experiment III: Anti-tumor efficacy of eIF5A1 gene therapy was improved by complexing DNA with DOTAP. 50,000 B16F10 cells were injected into the tail vein of 6-week old C57BL/6 mice (Day 0). Plasmid DNA bearing either the LacZ gene (as a DNA control) or eIF5A1 complexed with DOTAP prior to injection into the tail vein on days 7, 14, and 21. Tumor-free mice that received injections of DOTAP without plasmid DNA were used as a control for the effects of DOTAP. Mice were sacrificed at Day 25 and the lungs were removed and photographed.

FIG. 29 shows the results of experiment III: Anti-tumor efficacy of eIF5A1 gene therapy was improved by complexing DNA with DOTAP. 50,000 B16F11 cells were injected into the tail vein of 6-week old C57BL/6 mice (Day 0). Plasmid DNA bearing either the LacZ gene (as a DNA control) or eIF5A1 complexed with DOTAP prior to injection into the tail vein on days 7, 14, and 21. Tumor-free mice that received injections of DOTAP without plasmid DNA were used as a control for the effects of DOTAP. Mice were sacrificed at Day 25 and the lungs were removed and weighed.

FIG. 30 shows the results of experiment IV: Ad-eIF5A1 injection induces apoptosis in B16F0 and B16F10 tumors. 500,000 B16F0 or B16F10 cells were subcutaneously injected into the right flank of C57BL/6 mice. 1×10⁹ pfu Ad-5A1 in 50 μl of PBS was injected into tumors when the tumors reached about 4 mm in diameter (10-12 days after B16 cell injection). Mice were sacrificed after 48 hours and tumors were excised, fixed, and embedded in paraffin. Two sections for each cell tumor type (Ad-5A1-1 and Ad-5A1-2) were stained by TUNEL (Promega) according to the manufacturer's protocol. Negative control slides (Ad-5A1-neg) in which the TdT enzyme was left out of the TUNEL reaction were included for each cell type.

FIG. 31 shows the results of experiment V: Tumor growth was significantly retarded by injection with Ad-eIF5A1. C57BL/6 bearing subcutaneous B16-F10 were injected with either 1×10⁹ pfu of Ad-eIF5A1 or Ad-LacZ or an equivalent volume of buffer. The first day of treatment occurred when tumors reached an approximate diameter of 8 mm and was designated Day 0. Mice were injected every day for the first three days and then every other day thereafter until the mouse was sacrificed. Mice were sacrificed when the tumor size exceeded 15 to 16 mm in one dimension. There were three mice in each group. Group 1 mice (1-1,1-2, 1-3) were treated with Ad-eIF5A1, Group 2 mice (2-1, 2-2, 2-3) were injected with Ad-LacZ and Group 3 (3-1, 3-2, 3-3) mice received only buffer. Tumor size was measured every day using calipers and used to estimate tumor volume.

FIG. 32 shows the results of experiment V: Survival was significantly increased by injection with Ad-eIF5A1. C57BL/6 bearing subcutaneous B16-F10 were injected with either 1×10⁹ pfu of Ad-eIF5A1 or Ad-LacZ or an equivalent volume of buffer. The first day of treatment occurred when tumors reached an approximate diameter of 8 mm and was designated Day 0. Mice were injected every day for the first three days and then every other day thereafter until the mouse was sacrificed. Mice were sacrificed when the tumor size exceeded 15 to 16 mm in one dimension. There were three mice in each group. Group 1 mice (1-1,1-2, 1-3) were treated with Ad-eIF5A1; Group 2 mice (2-1, 2-2, 2-3) were injected with Ad-LacZ; and Group 3 (3-1, 3-2, 3-3) mice received only buffer.

FIG. 33 shows that eIF-5A1 increases the accumulation and phosphorylation of p53 tumor suppressor protein in A549 lung carcinoma cells.

FIG. 34 shows that eIF-5A1 increases p53 mRNA levels in A549 lung carcinoma cells.

FIG. 35 shows that the increase in p53 levels is dependent upon p53 transcriptional activity in A549 lung carcinoma cells.

FIG. 36 shows that TNFR1 mRNA levels are upregulated by infection with eIF-5A1 in A549 lung carcinoma cells.

FIG. 37 shows that eIF-5A1 induced apoptosis in A549 lung carcinoma cells and the use of a MEK inhibitor increases the amount of apoptosis induced by eIF-5A1.

FIG. 38 shows a hypothetical model of eIF-5A1s effect on MAPK/SAPK pathways and apoptosis in A549 lung carcinoma cells.

FIG. 39 shows that eIF-5A1 is better than eIF-5A2 in suppression of tumor growth (in mouse xenograft model (B16-FO melanoma cells)).

FIG. 40 shows that eIF-5A1 is better than eIF-5A2 in prolonging mouse survival (in mouse xenograft model (B16-FO melanoma cells)).

FIG. 41 shows that eIF-5A increases the number of dead or dying cells as compared to cells being treated with the control with or without IL-6.

FIG. 42 provides the sequence of eIF-5A2.

FIG. 43 provides the location and sequences of eIF-5A1 siRNAs.

FIG. 44 provides a nucleotide alignment of human eIF-5A1 with human eIF-5A2.

FIG. 45 provides an amino acid alignment of human eIF-5A1 with human eIF-5A2.

FIGS. 46 A and 46 B provides the location and sequences of eIF-5A1 siRNAs.

### DETAILED DESCRIPTION OF THE INVENTION

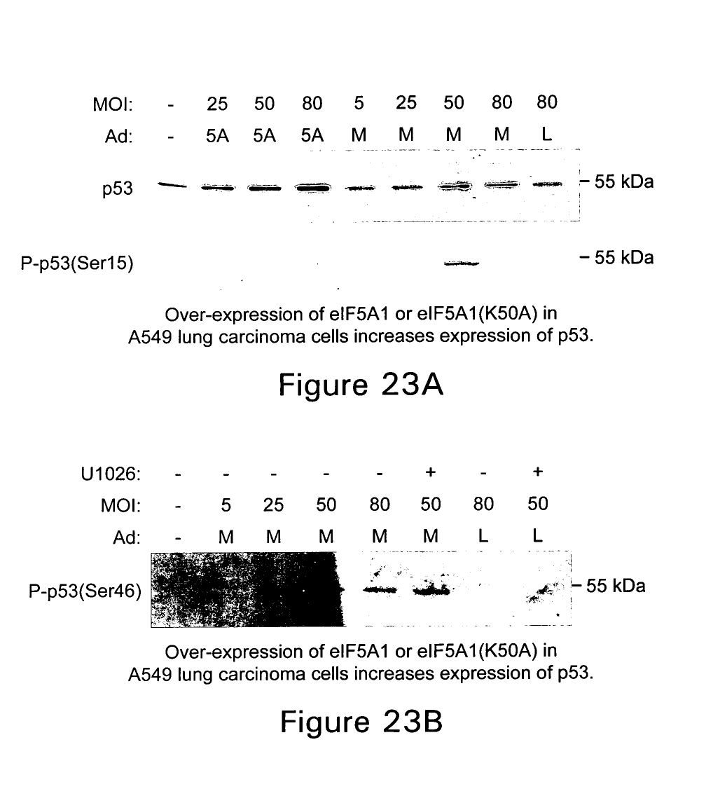

Eukaryotic translation initiation factor 5A1 (eIF5A1) has been hypothesized to function as a nucleocytoplasmic shuttle protein involved in facilitating translation of subsets of mRNAs involved in cell proliferation. However, eIF5A1 has also been identified as a regulator of apoptosis (Taylor et al., Invest Ophthalmol Vis Sci.; 45(10):3568-76 (2004)) and a pro-apoptotic protein capable of regulating expression of p53 (Li et al. (2004) J. Biol. Chem.; 279:49251-49258; and FIG. 23 of this document). The tumor suppressor protein p53 plays a central role in mediating cell cycle arrest and apoptosis in response to stress and DNA damage. Although over-expression of eIF5A1 is capable of up-regulating p53 expression in cancer cell lines (Li et al. (2004) J. Biol. Chem.; 279:49251-49258; and FIG. 23 of this document), over-expression of eIF5A1 is also capable of inducing apoptosis in p53-deficient cell lines (Taylor et al. (2006) Journal of Molecular and Cellular Biology, Feb. 9, 2006 (decision pending)) indicating that eIF5A1 over-expression induces apoptosis by multiple mechanisms.

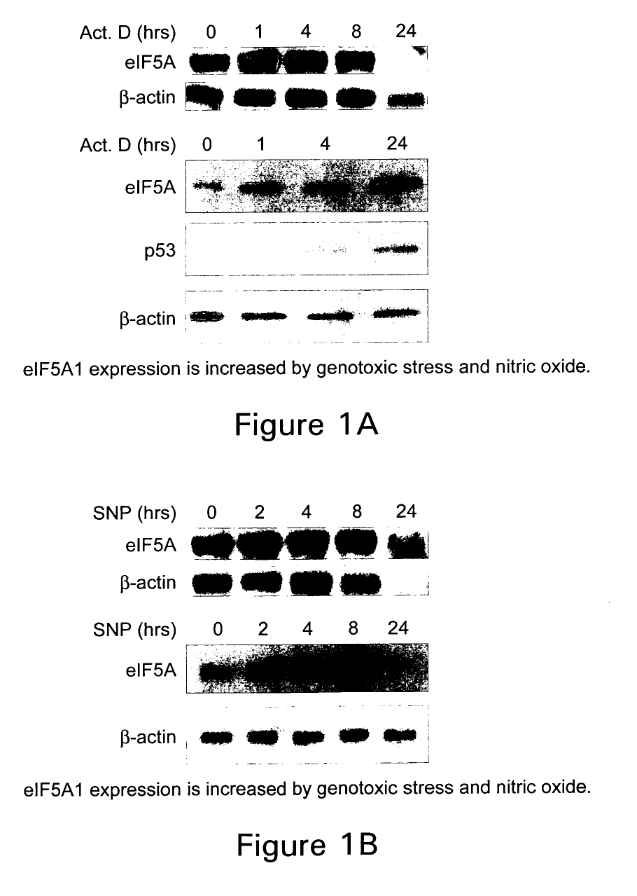

Expression of eIF5A1 is correlated to apoptosis. Western blots of normal colon fibroblasts treated with the topoisomerase inhibitor Actinomycin D (FIG. 1A) demonstrate that eIF5A1 protein expression is up-regulated in parallel with p53 twenty four hours after initiation of Actinomycin D treatment. RKO cells undergoing apoptosis resulting from exposure to the nitric oxide (NO)-donor, SNP, also upregulate eIF5A1 protein expression (FIG. 1B). RNA transcript levels for eIF5A1 do not increase under these conditions indicating that eIF5A1 protein is accumulating as a result of post-transcriptional regulation of mRNA (FIGS. 1A and 1B). These results indicate that eIF5A1 may be involved in apoptosis resulting from genotoxic stress or nitric oxide.

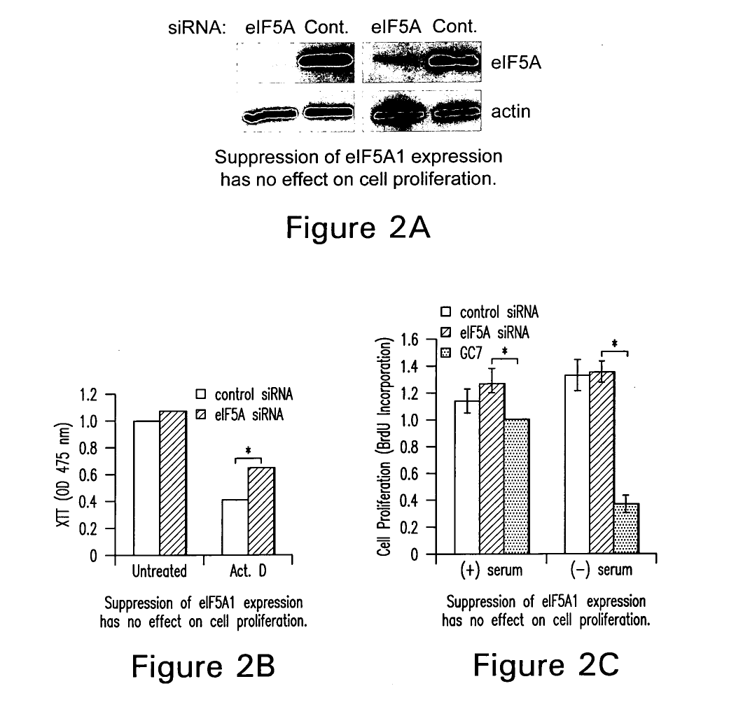

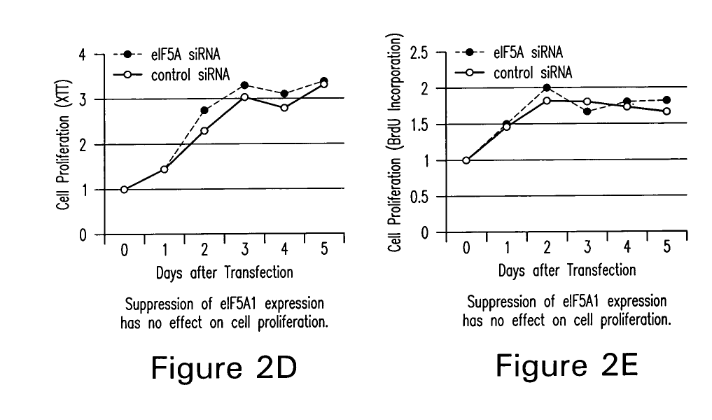

A requirement for eIF5A1 in cell proliferation has long been proposed, partly due to reports of DHS and DHH inhibitors inducing cell cycle arrest and apoptosis and the conclusion that hypusinated eIF5A1 must be required to sustain cell growth. However, it was determined that specific suppression of eIF5A1 expression through the use of siRNAs had no effect on cell growth. Suppression of eIF5A1 expression by >90% in HT-29 cells had no effect on proliferation of the cells over a period of 5 days (FIG. 2A-E). The DHS inhibitor GC7, however, had a profound effect on the growth of these cells (FIG. 2C). These results suggest that eIF5A1 may not be required for cell growth. Also of interest is the fact that suppression of eIF5A1 was able to partially protect HT-29 cells from Actinomycin D-induced cytotoxicity (FIG. 2B), further supporting eIF5A1's involvement in apoptosis resulting from genotoxic stress.

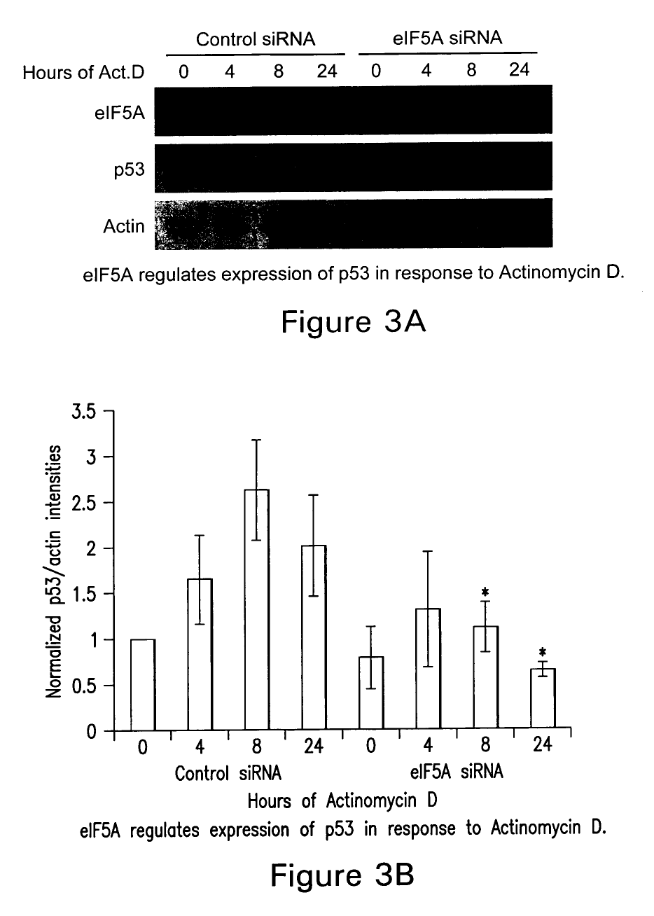

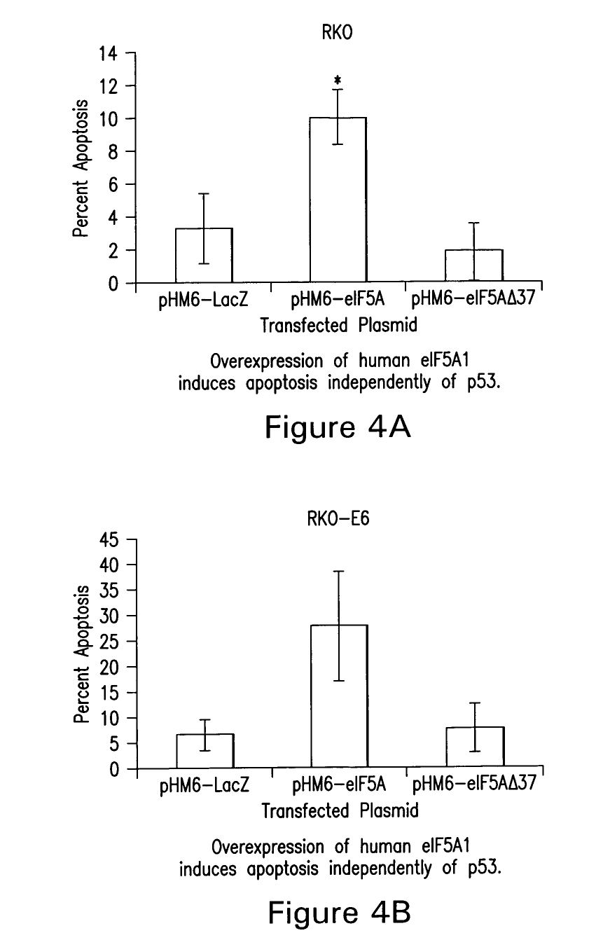

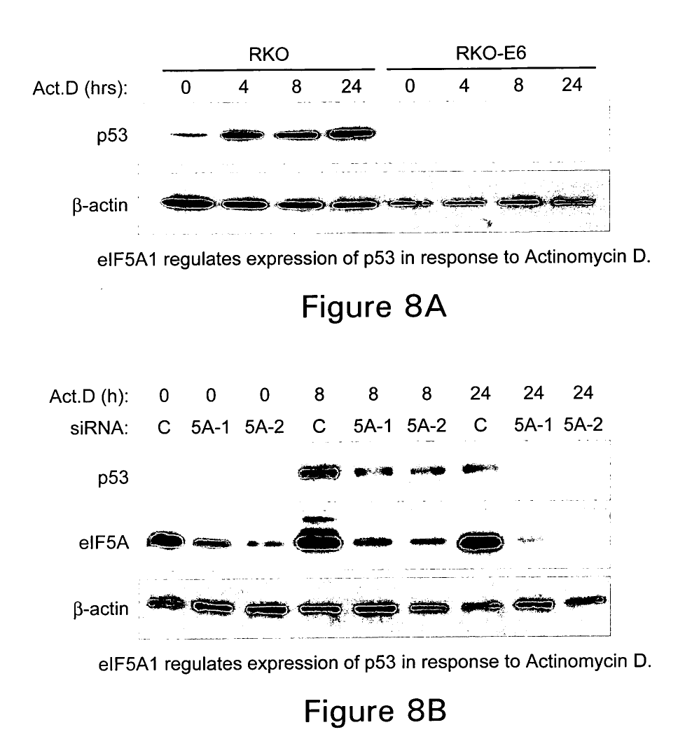

The ability of eIF5A1 siRNAs to protect cells from apoptosis prompted the examination of the effect eIF5A1 protein suppression had on p53 expression. RKO cells have a functional p53 protein that does not accumulate except under conditions of stress. Suppression of eIF5A1 by siRNA was able to inhibit the accumulation of p53 protein by 69% after twenty fours of Actinomycin D treatment (FIGS. 3A and 3B), indicating that eIF5A1 is required for proper expression of p53 during genotoxic stress. A second siRNA against eIF5A1 having a different sequence was also able to prevent p53 upregulation in response to Actinomycin D (FIG. 8B) indicating that this effect is not a non-specific effect of the siRNA. However, eIF5A1 was capable of inducing apoptosis in both RKO cells (with functional p53) and RKO-E6 cells (without functional p53; FIG. 8A) (FIGS. 4A and 4B), indicating that eIF5A1 can induce apoptosis by p53-independent mechanisms as well.

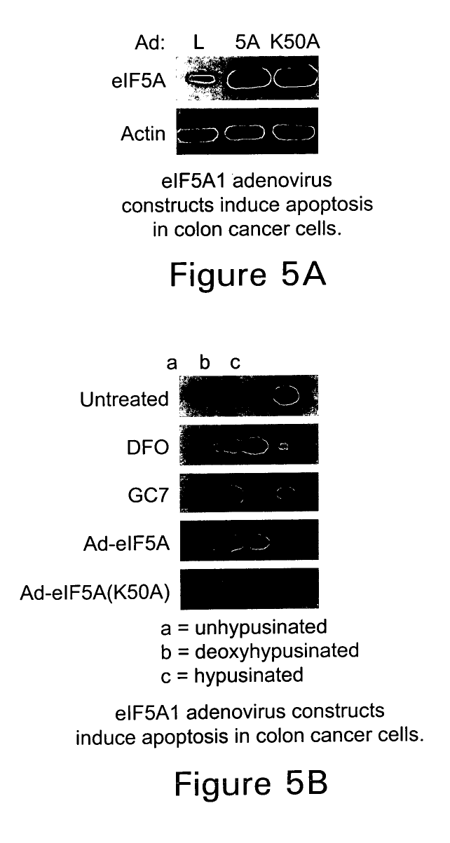

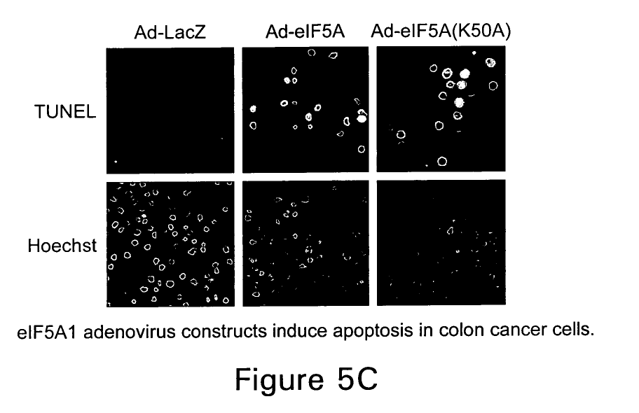

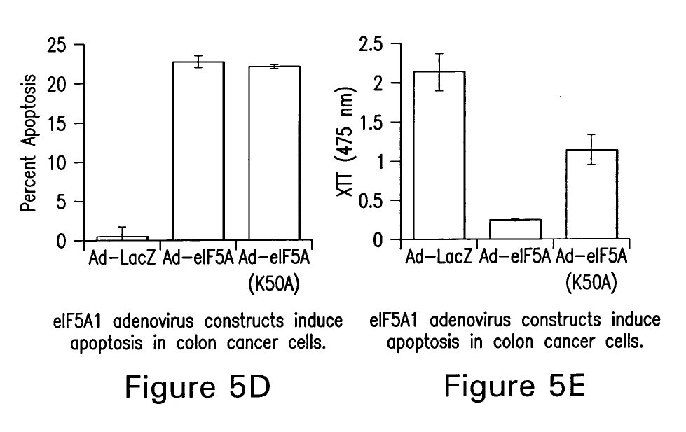

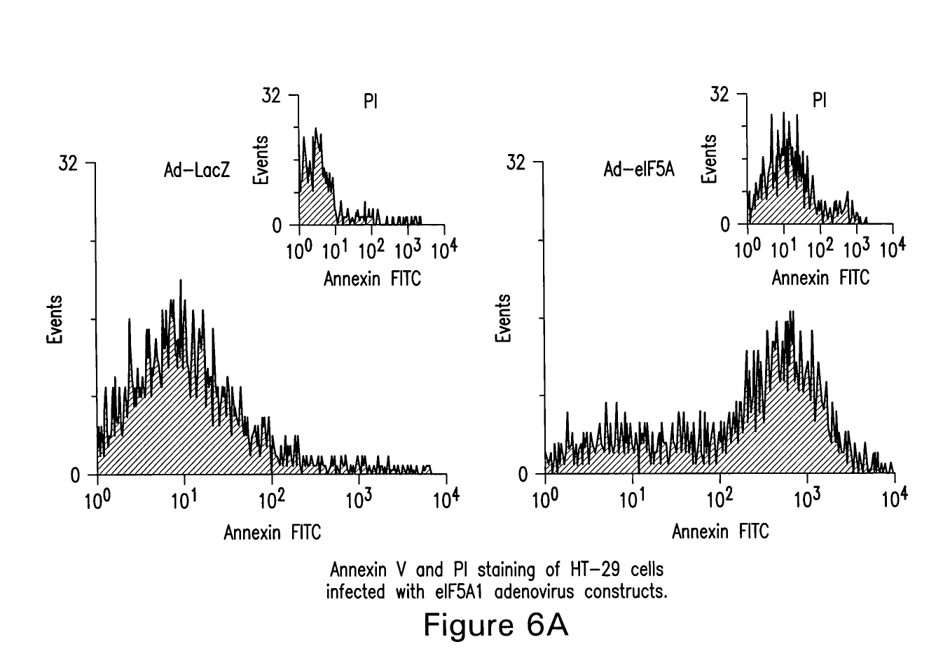

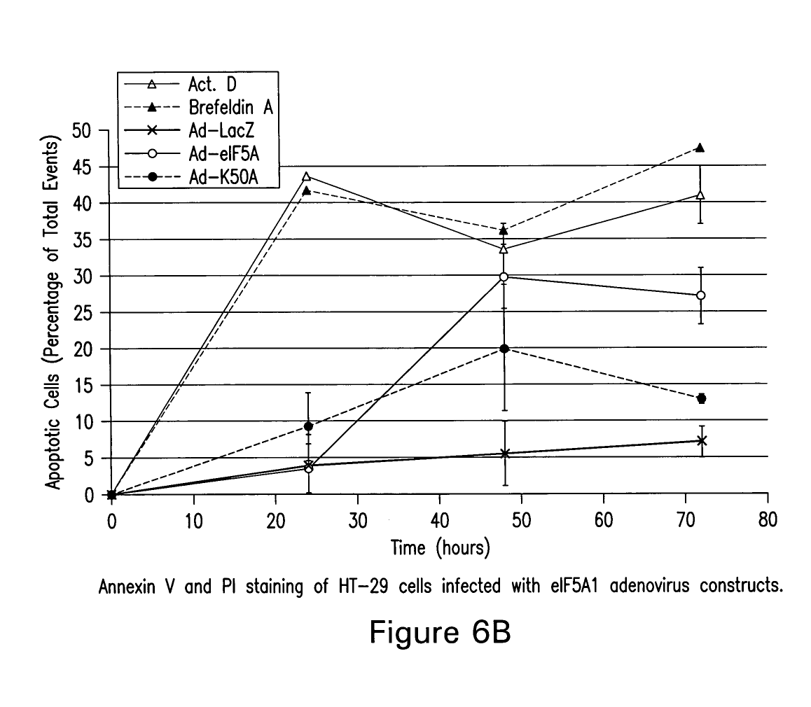

Adenovirus constructs expressing either eIF5A1 or eIF5A1 containing a point mutation in the conserved lysine (K)(position 50) that is required for the hypusine modification {eIF5A1(K50A)}were constructed. The point mutation caused the lysine to be an alanine (A). Infection of HT-29 cells with either construct induced apoptosis in these cells (FIGS. 5C and 5D and FIG. 6) and greatly decreased cell viability (FIG. 5E). Two dimensional gel electrophoresis was used to determine which form of eIF5A1 was accumulating as a result of infection with Ad-eIF5A1 or Ad-eIF5A1(K50A) (FIG. 5B). As expected, unmodified eIF5A1 accumulated after Ad-eIF5A1(K50A) infection. Infection with Ad-eIF5A1 resulted in a dramatic increase in the accumulation of both unmodified and deoxyhypusine-modified eIF5A1, indicating that DHS and DHH activities were insufficient to hypusinate most of the eIF5A1 protein being generated by the virus (FIG. 5B). These results strongly suggest that the unmodified eIF5A1 and perhaps the deoxyhypusine-modified eIF5A1 are the forms leading to apoptosis of the cells. It is not yet clear whether hypusinated eIF5A1 shares this ability.

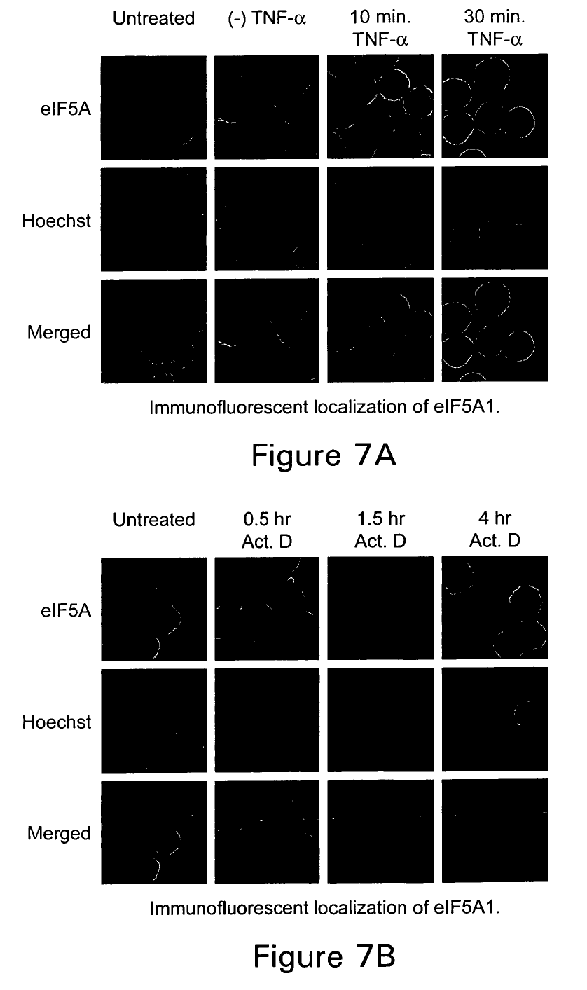

A nucleocytoplasmic shuttling function has been proposed for eIF5A1, however, eIF5A1 has been reported to be expressed predominantly in the cytoplasm (Shi et al., Exp Cell Res.; 225:348-356 (1996b)). A nucleocytoplasmic shuttle protein would be expected to have a nuclear localization as well. Since a function for eIF5A1 during apoptosis was found, the localization of eIF5A1 changes during apoptosis were studied. Apoptosis was induced in HT-29 cells by two different mechanisms, death receptor activation via treatment with IFN-γ and TNF-α and genotoxic stress via treatment with Actinomycin D. Indirect immunofluorescence revealed that eIF5A1 localization was cytoplasmic in untreated, growing cells (FIG. 7). However, treatment with apoptotic stimulators very quickly stimulated the movement of eIF5A1 from a predominantly cytoplasmic localization to a predominantly nuclear localization (FIGS. 7A and 7B). This shift in eIF5A1 localization occurred very quickly, within 10 minutes for IFNγ/TNF-α-treated cells (FIG. 7A) and within 90 minutes for cells treated with Actinomycin D (FIG. 7B). These results indicate that eIF5A1 has a nuclear function during apoptosis induced by both death receptor activation and genotoxic stress.

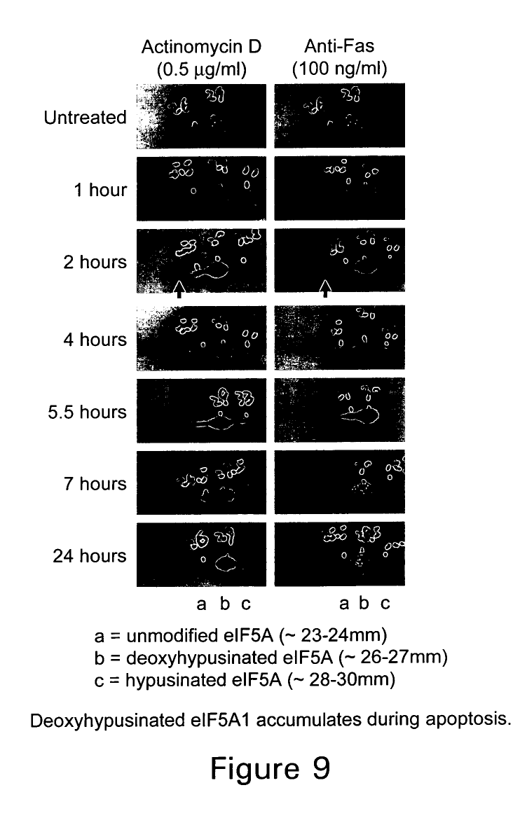

In order to clarify whether it is the unmodified, deoxyhypusine-modified, or hypusine-modified form of eIF5A1 that is involved in apoptosis, the forms of eIF5A1 accumulating during apoptosis was examined by 2-D gel electrophoresis. HT-29 cells were induced to undergo apoptosis by stimulation with either Actinomycin D (genotoxic stress) or incubation with an agonistic antibody against Fas (death receptor pathway). Cell lysates were collected after various time points ranging from 1 hour to 24 hours and then analyzed by 2-D gel electrophoresis and Western blotting with eIF5A antibody (FIG. 9). In untreated cells, the eIF5A1 protein was predominantly in the hypusinated form in agreement with what is reported in the literature. Both apoptotic inducers stimulated an increase in the presence of the deoxyhypusinated form of eIF5A1 beginning 1 hour after treatment but disappearing 24 hours after treatment. An accumulation of unmodified eIF5A1 was also observed 2 hours after treatment. These results are consistent with the deoxyhypusinated and/or unmodified eIF5A1 being involved in regulating apoptosis.

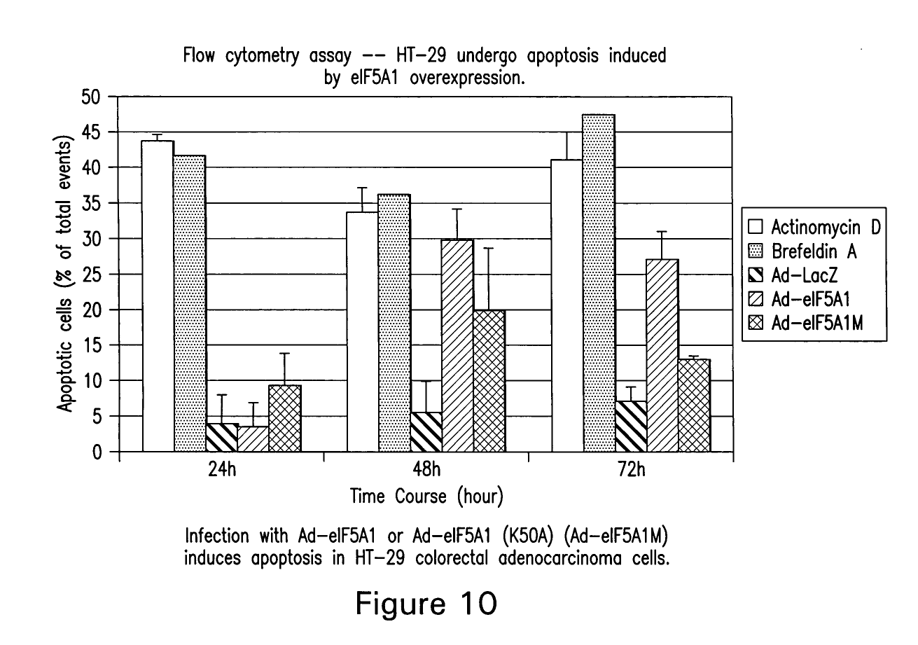

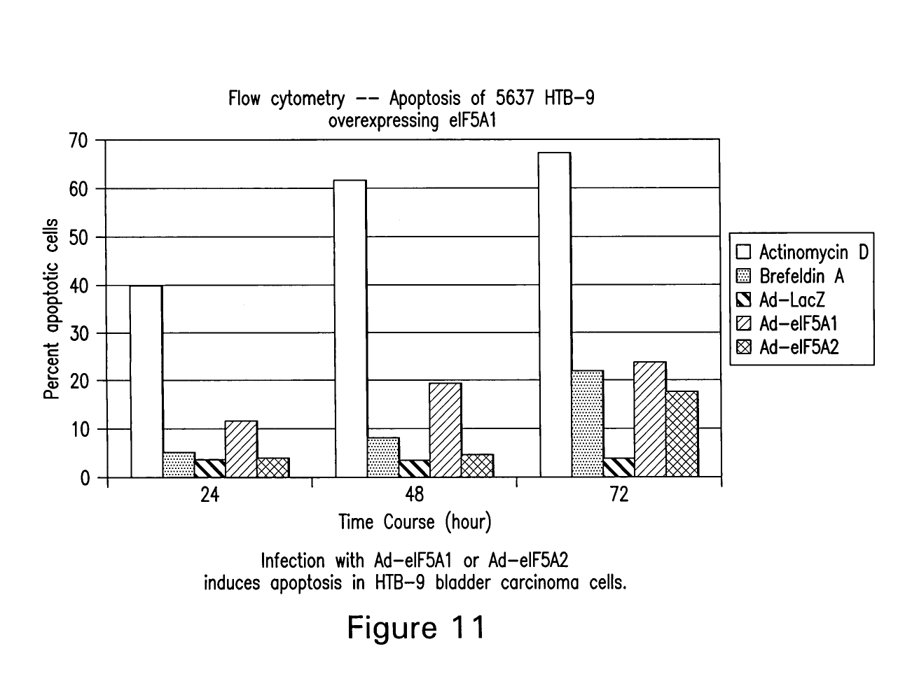

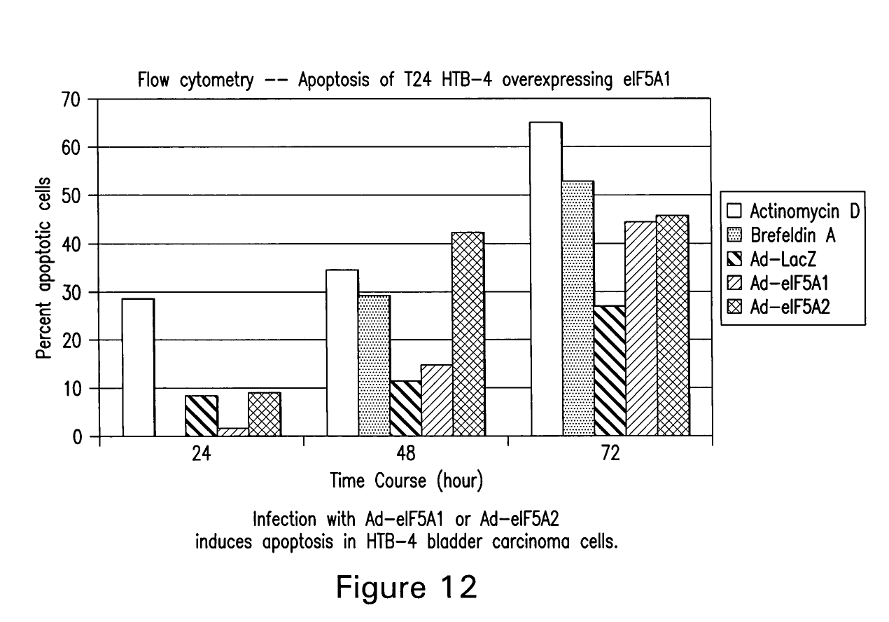

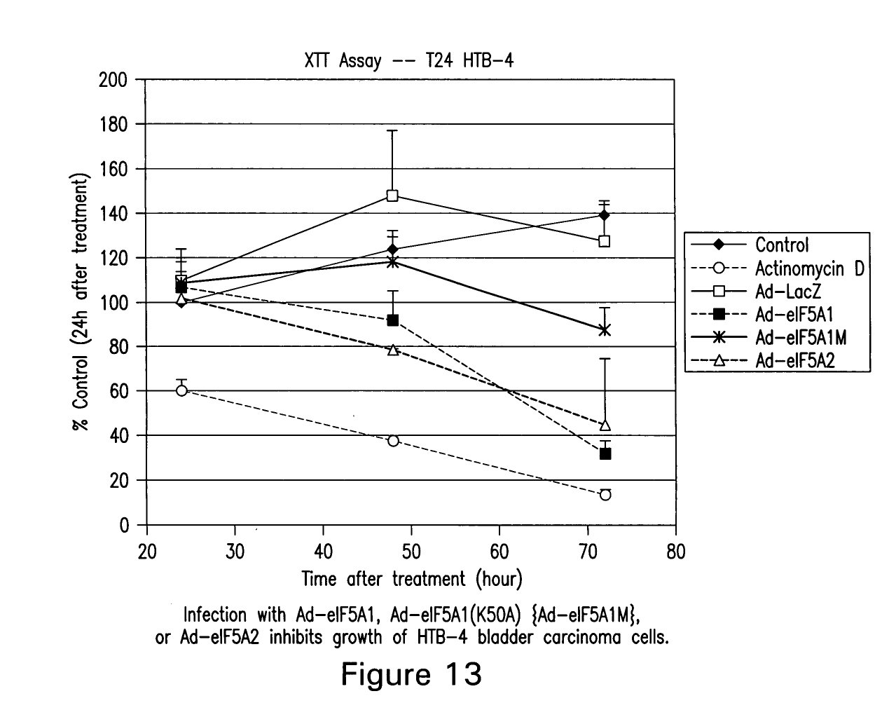

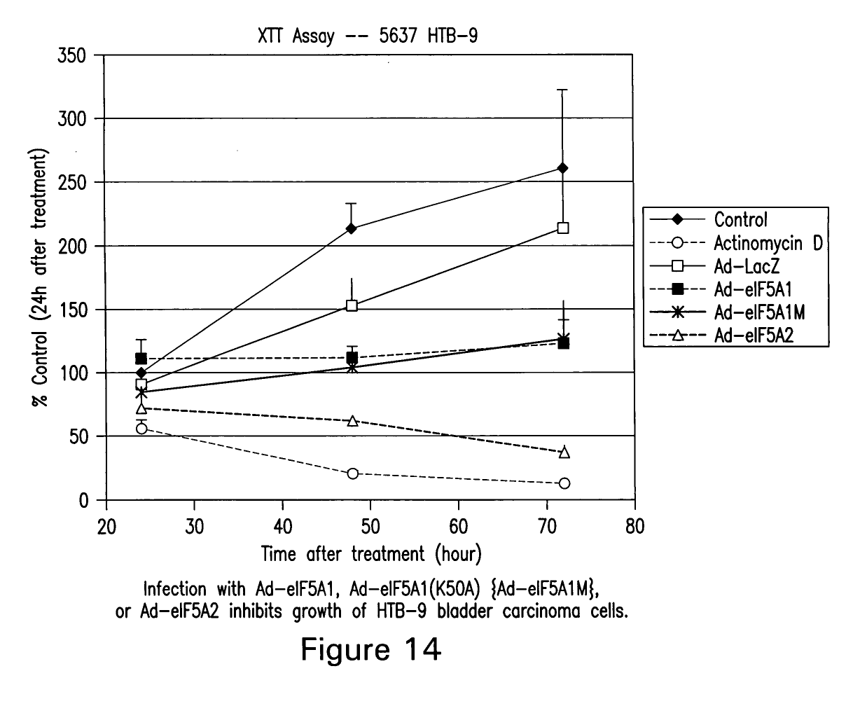

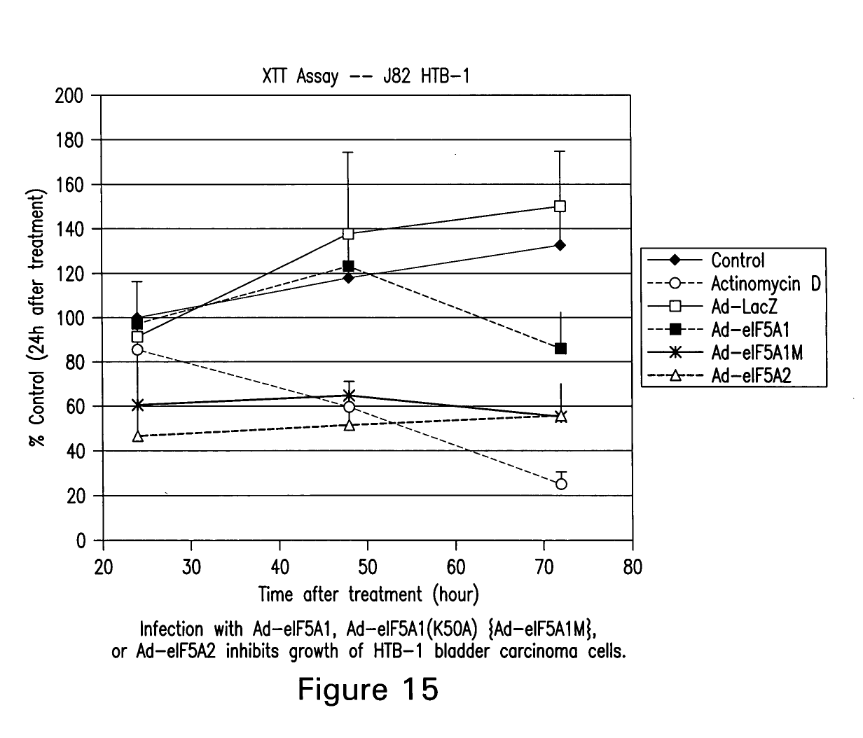

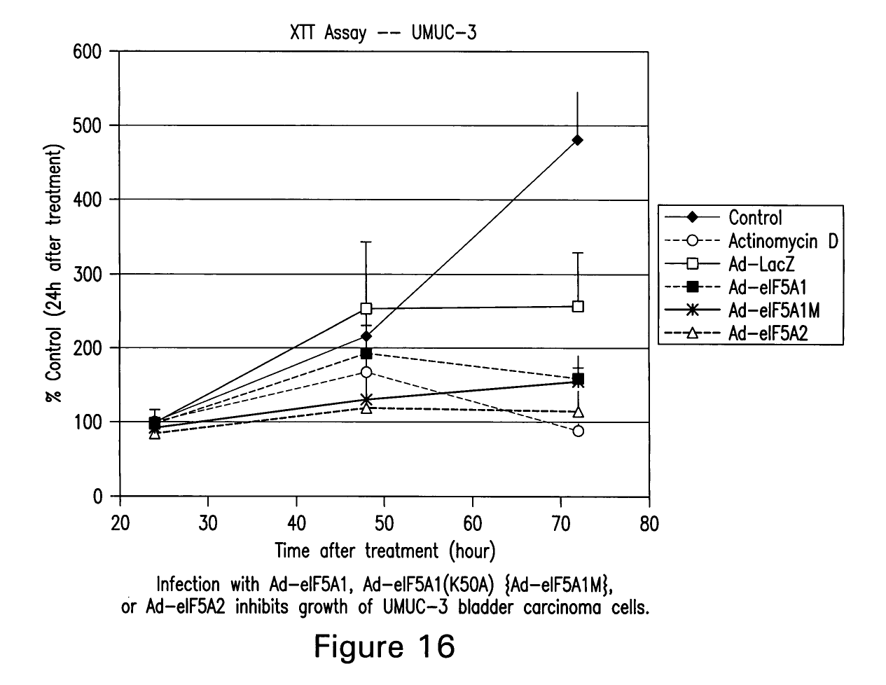

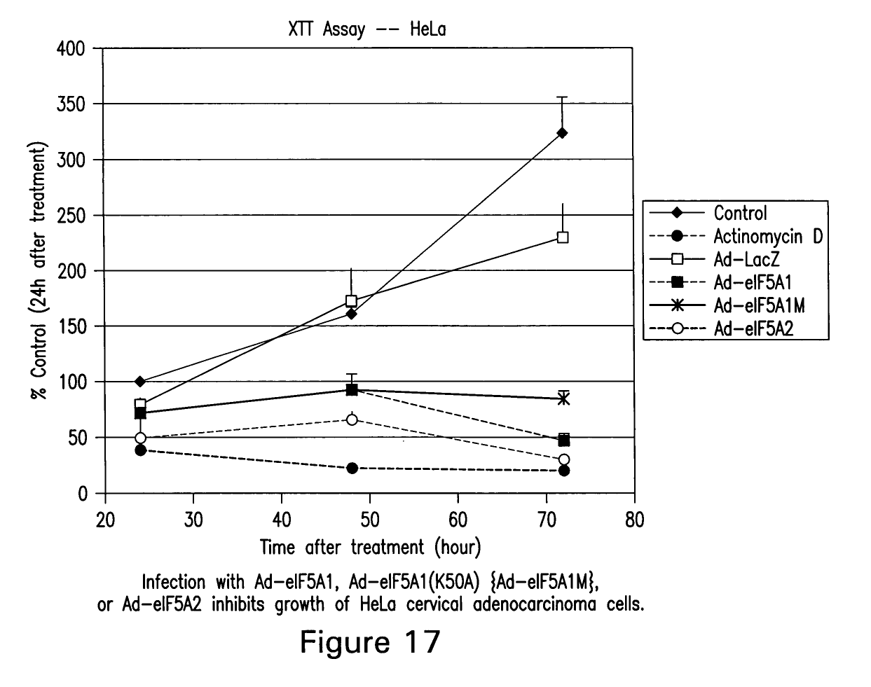

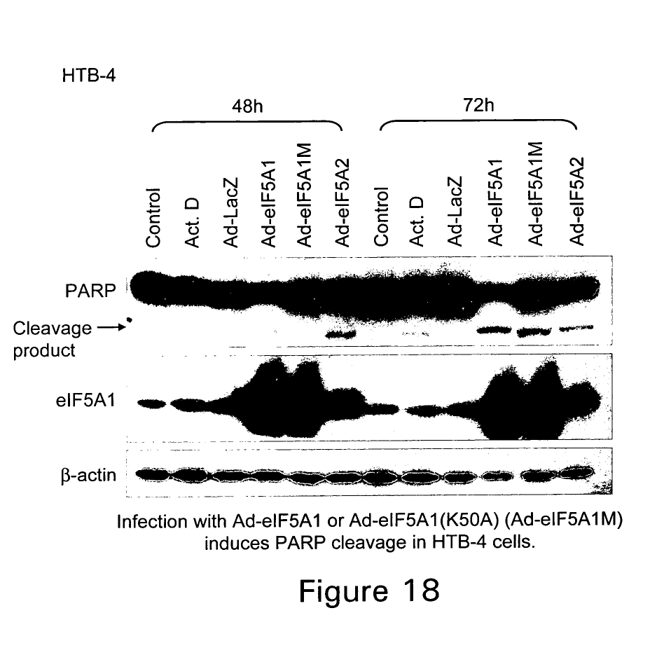

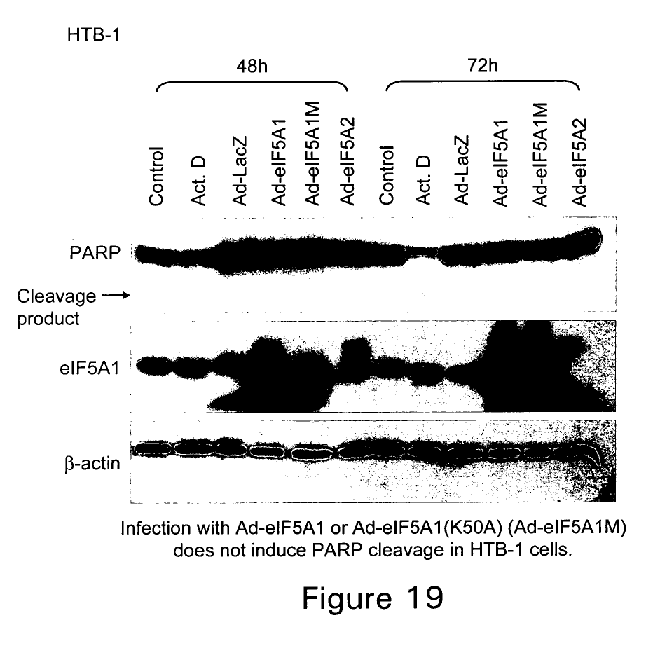

The ability of eIF5A1, eIF5A1(K50A)(mutant eIF5A1), and eIF5A2 to induce apoptosis and to inhibit proliferation was examined in a variety of cancer cell lines. Both eIF5A1 and eIF5A1(K50A) were able to induce apoptosis in the colon carcinoma cell line HT-29 (FIGS. 5, 6, and 10) and the bladder cancer cell lines HTB-9 (FIG. 11) and HTB-4 (FIG. 12). In addition, infection with an adenovirus expressing eIF5A2 can induce apoptosis in the bladder cancer cell lines HTB-9 (FIG. 11) and HTB-4 (FIG. 12). Ad-eIF5A1, Ad-eIF5A1(K50A), and Ad-eIF5A2 were all able to inhibit growth of the bladder cancer cell lines HTB-4 (FIG. 13), HTB-9 (FIG. 14), J82 HTB-1 (FIG. 15), and UMUC-3 (FIG. 16). These viral constructs were also able to inhibit growth of HeLa cervical adenocarcinoma cells (FIG. 17). Apoptosis in HTB-4 cells was also observed by PARP cleavage in response to Ad-eIF5A1, Ad-eIF5A1(K50A), and Ad-eIF5A2 infection (FIG. 18). PARP, which is normally involved in DNA repair, DNA stability and other cellular events, is cleaved by members of the caspase family during early apoptosis. Detection of caspase cleavage of PARP has been shown to be a hallmark of apoptosis. Lazebnik Y, et al., Nature 371, 346-347 (1994). These results indicate that eIF5A1 and eIF5A2 may in fact have redundant functions during apoptosis. Furthermore, the mutant form of eIF5A1 was able to arrest growth and induce apoptosis a variety of cell lines. The ability of the wild-type eIF5A1 (and eIF5A2) to induce apoptosis could be related to the accumulation of deoxyhypusinated and unmodified eIF5A1 that occurs (FIG. 5B) when Ad-eIF5A1 is introduced into a cell due to limiting activities of DHS and DHH. A recent report has shown that in order for exogenous eIF5A1 to be over-expressed in a cell in the hypusinated form, both DHS and DHH have to be over-expressed in the same cell (Park et al. 2006, PNAS; 103(1): 51-56). These results suggest that the ability of DHS and DHH inhibitors to inhibit cell growth may not be due a decrease in hypusinated eIF5A1 required for cell proliferation, but instead may be due to an accumulation of unmodified (DHS inhibitors) or deoxyhypusine-modified (DHH inhibitors) eIF5A1, which in turn trigger cell cycle arrest and/or apoptosis in the cell. It is also interesting to note that DHS has been found to be over-expressed in certain cell lines (Clement et al. 2006, FEBS J; 273(6):1102-14) and that DHS has been identified as one of a signature set of amplified genes in cancer metastases (Ramaswamy et al. 2003, Nat Genet; 33, 49-54.). One possible interpretation of this information is that certain cancer cells over-express DHS in order to prevent apoptosis triggered by accumulation of unmodified or deoxyhypusinated eIF5A1 (which appear to accumulate when cells are triggered to undergo apoptosis, see FIG. 9). By over-expressing DHS, cancer cells may be able to reduce apoptosis caused by genotoxic stress and other stressors by keeping eIF5A1 in a hypusinated and thereby, “safe” form.

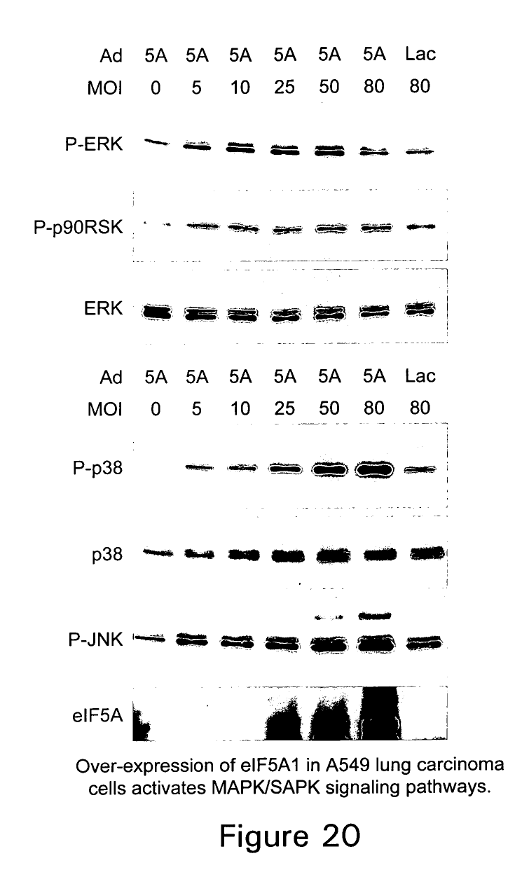

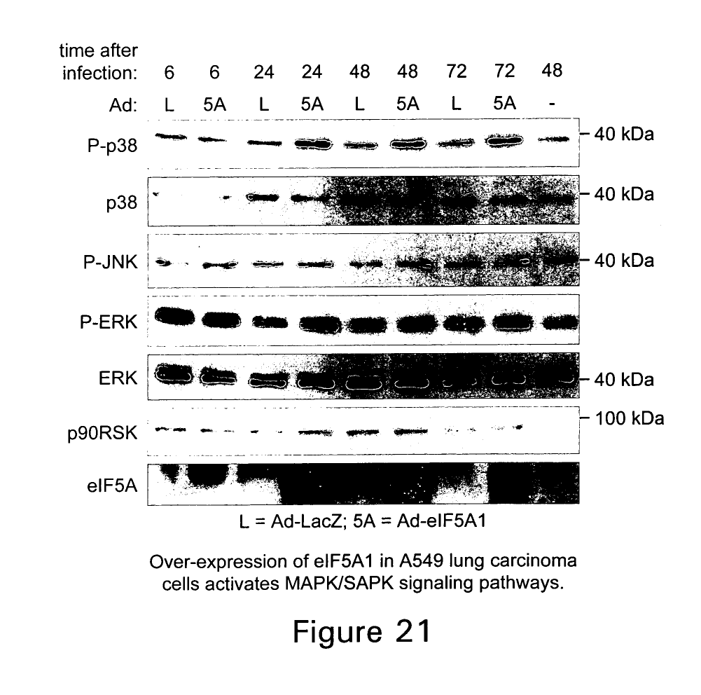

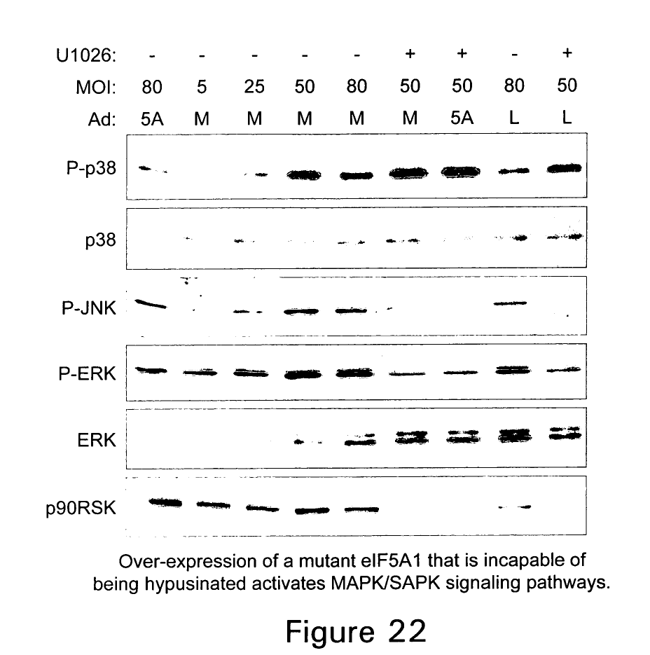

In order to elucidate which signaling pathways are affected by over-expression of eIF5A1, activation of the mitogen activated protein kinase (MAPK)/stress activated protein kinase (SAPK) [MAPK/SAPK] pathways was examined in response to Ad-eIF5A1 or Ad-eIF5A1(K50A) infection in A549 lung carcinoma cells. The three major MAPK pathways are the ERK MAPK pathway, the p38 MAPK pathway, and the JNK SAPK pathway. The ERK MAPK pathway is mainly triggered in response to mitogenic stimuli such as growth hormones like epidermal growth factor (EGF) and supports the growth and survival of a broad range of tumors. The p38 MAPK pathway is activated in response to cellular stresses, UV light, growth factor withdrawal, and pro-inflammatory cytokines. Activation of p38 via phosphorylation leads to the phosphorylation of transcription factors such as p53, which can in turn lead to increased activity or stability of p53. Activation of p38 is involved in both pro-apoptotic and anti-apoptotic pathways as well as inflammation. The JNK/SAPK pathway mediates responses to cellular stresses including UV light, DNA damage and pro-inflammatory cytokines and results in the phosphorylation and increased activity of transcription factors such as c-jun. Activation of the JNK pathway can lead to numerous cellular responses including growth, transformation and apoptosis. The JNK pathway appears to be a prime effector pathway for EGF-induced growth in A549 cells (Bost et al. 1997, JBC; 272:33422-33429). Infection of A549 cells with Ad-eIF5A1 induces the activation of all three of these pathways. Infection with increasing amounts of Ad-eIF5A1 in A549 cells stimulated with EGF for 30 minutes, resulted in increasing phosphorylation/activation of ERK and its downstream target, p90RSK, while amounts of unphosphorylated ERK remained unchanged (FIG. 20). A strong upregulation of phosphorylated/activated p38 and phosphorylated/activated JNK was also observed in response to Ad-eIF5A1 infection in a dose responsive manner (FIG. 20). A time course of Ad-eIF5A1 infection revealed that activation of ERK, p38, and JNK pathways was sustained from 24 to 72 hours after infection (FIG. 21). A dose-responsive increase in activation of these pathways in response to infection with the unhypusinated mutant eIF5A1, Ad-eIF5A1(K50A) (FIG. 22) was found. It is of interest to note that inhibiting the activation of ERK using a MEK1 inhibitor, U1026, also inhibited the activation of JNK (FIG. 22), confirming that in these cells, JNK is activated in response to ERK activation (Bost et al. 1997, JBC; 272:33422-33429). In contrast, the MEK1 inhibitor has consistently resulted in increased p38 activation in response to either Ad-eIF5A1 or Ad-eIF5A1(K50A) infection indicating that activation of the ERK pathway negatively regulates activation of the p38 pathway in response to eIF5A1 (FIG. 22).

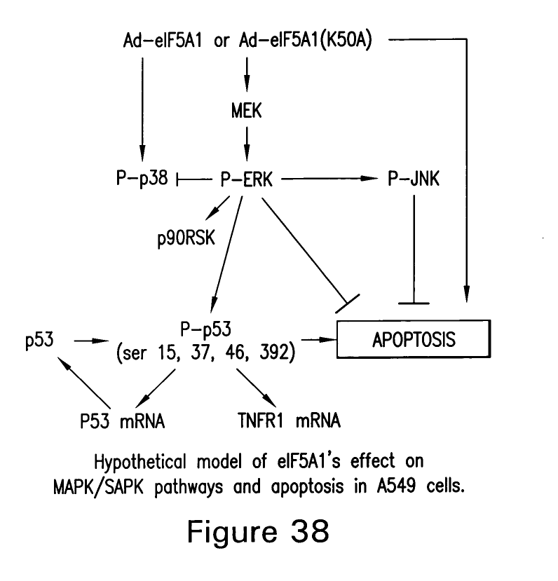

FIG. 38 provides a hypothetical model of eIF-5A1's effect on MAPK/SAPK pathways and apoptosis in A549 lung carcinoma cells. Over-expression of eIF5A1 (either wild-type or a mutant incapable of being hypusinated)(K50A) induces apoptosis in A549 cells. Over-expression of eIF5A1 is also accompanied by an increase in MAPK/SAPK pathways, including p38, JNK, and ERK. Over-expression of eIF5A1 induces an increase in p53 protein levels, including increases in phosphorylated forms of p53 that are associated with increased stability and activity of p53. This increase in p53 is dependent on the activity of the MEK/ERK pathway and on p53 transcriptional activity. However, inhibition of the MEK/ERK pathway, and to a lesser extent inhibition of the JNK pathway, results in a potentiation of the apoptosis induced by eIF5A1. These results indicate that eIF5A1 may have use as a therapeutic in order to induce apoptosis in cancer cells as well as to increase the cancer cell-killing abilities of the MEK inhibitor class of anti-cancer drugs.

The effect of Ad-eIF5A1 and Ad-eIF5A1(K50A) on the expression and phosphorylation of p53 can be seen in FIG. 23. Over-expression of both wild-type and mutant eIF5A1 led to an increase in the overall expression of p53 protein, in a dose-responsive manner (FIG. 23). An increase in p53 phosphorylated on serine 15 was also observed for both forms of eIF5A1 (FIG. 23). An increase in p53 phosphorylated on serine 46 was observed in a dose-responsive manner for Ad-eIF5A1(K50A). p53 can be phosphorylated on serine 15 by several kinases including ATM, ATR, and p38, and this modification reduces the ability of MDM2 to bind p53 and target it for ubiquination and thereby promotes the accumulation and functional activity of p53. p53 is phosphorylated at serine 46 by various kinases including PKCdelta and p38 and this modification increases the affinity of p53 for pro-apoptotic promoters and is believed to be important for p53-induced apoptosis.

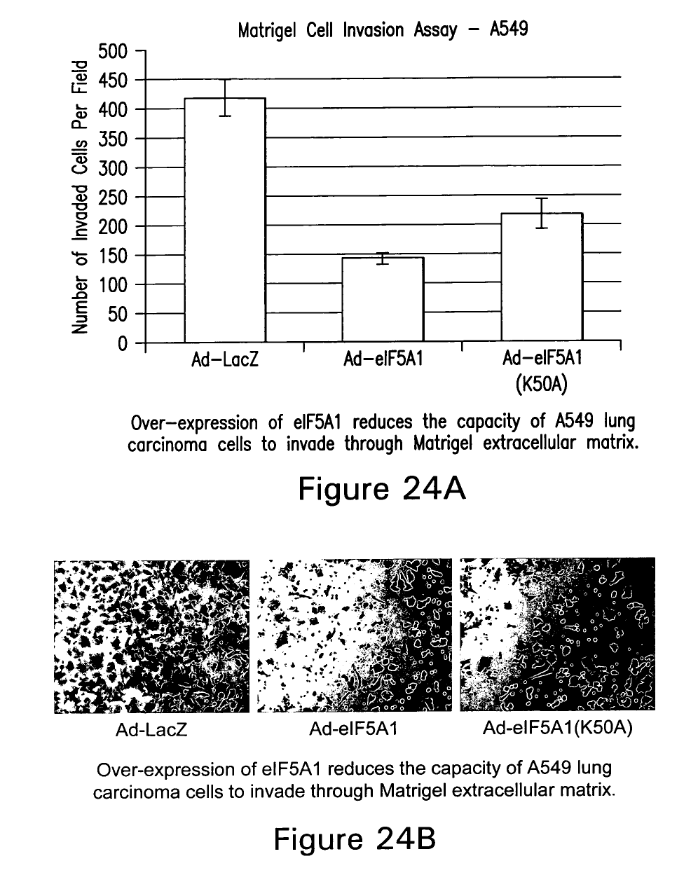

Invasion and metastasis of tumors is a complex process that requires a tumor cell to adapt its ability to adhere, to degrade the surrounding extracellular matrix, to migrate and proliferate at a secondary site, and finally to promote angiogenesis to sustain increased growth. Basement membranes are contiguous sheets of extracellular matrix (ECM) that surround every organ and act as a barrier to macromolecules and cells. The invasiveness of tumor cells can be measured by coating transwells with an 8 μm membrane with reconstituted ECM (Matrigel™) and staining cells that are able to penetrate this layer and reach the other side of the membrane in response to chemotactic stimulation. A549 lung carcinoma cells are highly invasive and are able to secrete proteins such as matrix metaloproteases, which are gelatinases capable of digesting components of the ECM. The effect of eIF5A1 over-expression on the invasiveness of A549 cells was examined—infection of Ad-eIF5A1 or Ad-eIF5A1(K50A) significantly decreased the number of cells that invaded through Matrigel™-coated transwells (FIG. 24).

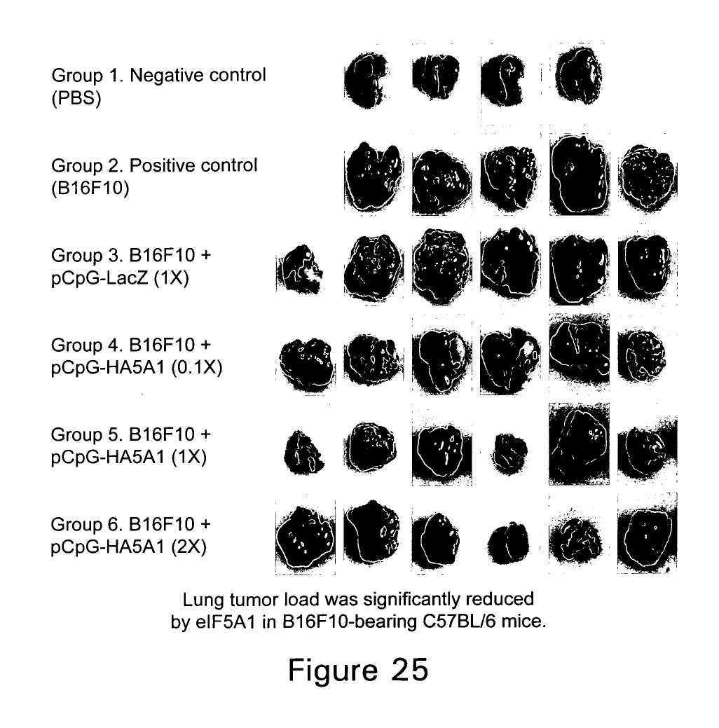

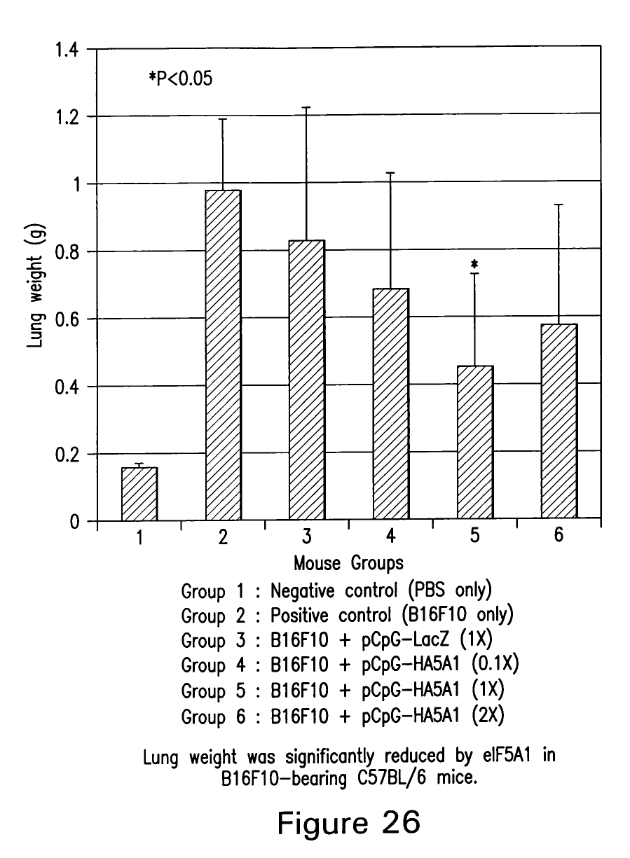

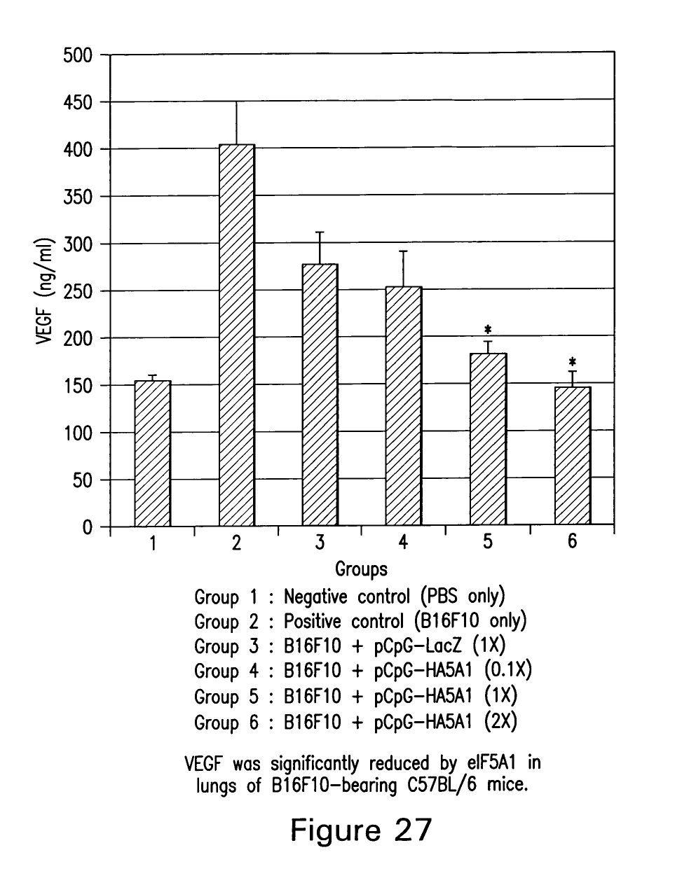

The results presented thus far suggested the possibility that treatment of tumors with eIF5A1 may be of therapeutic benefit. Thus, a model of experimental metastasis in mice was used. In Experiment II, experimental metastasis was initiated by injecting mice with the highly invasive mouse melanoma cell line B16F10 (day 0). Plasmid DNA encoding either the LacZ gene (as a DNA control) or eIF5A1 was injected into the tail vein day on days 2, 4, 7, 11, 16, 21, 26, and 31. Three different concentrations of DNA were used: 1× (3.3 mg/kg), 0.1× (0.3 mg/kg), and 2× (6.6 mg/kg). When the mice became moribund, they were sacrificed and the lungs were removed and photographed (FIG. 25). A dose-responsive decrease in tumor burden is observed when mice are treated with plasmid DNA encoding eIF5A1 (FIGS. 25 and 26). Although the 2× dose appeared to be less effective than the 1× dose, there was a significant decrease in tumor burden observed between the 0.1× dose and the 1× dose. The ability of tumors to grow macroscopically is dependent on the formation of new blood vessels (angiogenesis). Vascular endothelial growth factor (VEGF) is a cytokine that is secreted by many tumor cells and is a key factor in promoting tumor angiogenesis. The cell lysate from B16F10-bearing lungs isolated in Experiment II were analyzed for VEGF expression by ELISA (FIG. 27). There was a significant, dose-responsive decrease in VEGF levels in mice that received eIF5A plasmid DNA, indicating that eIF5A1 may regulate VEGF.

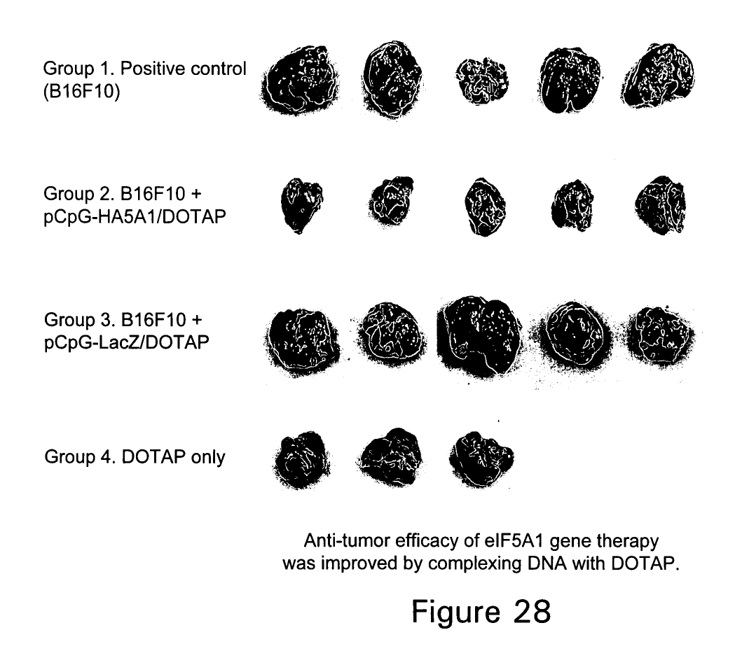

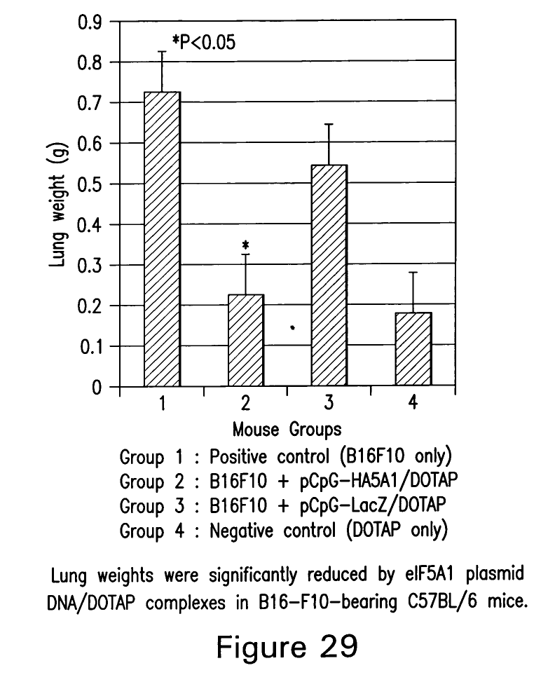

In Experiment III, a model of experimental metastasis using tail vein injected B16F10 cells was again used, but this time the plasmid DNA was complexed with DOTAP in order to increase the half-life of the DNA in serum and increase uptake of the plasmid into the lung tumors. Injections of DNA/DOTAP complexes occurred on Days 7, 14, and 21. Mice were sacrificed when they became moribund and the lungs were removed and photographed (FIG. 28). There was an average 60% reduction in lung weight in mice treated with the eIF5A1 plasmid DNA/DOTAP complexes (FIG. 29) when compared to mice that received the LacZ plasmid DNA/DOTAJP complexes.

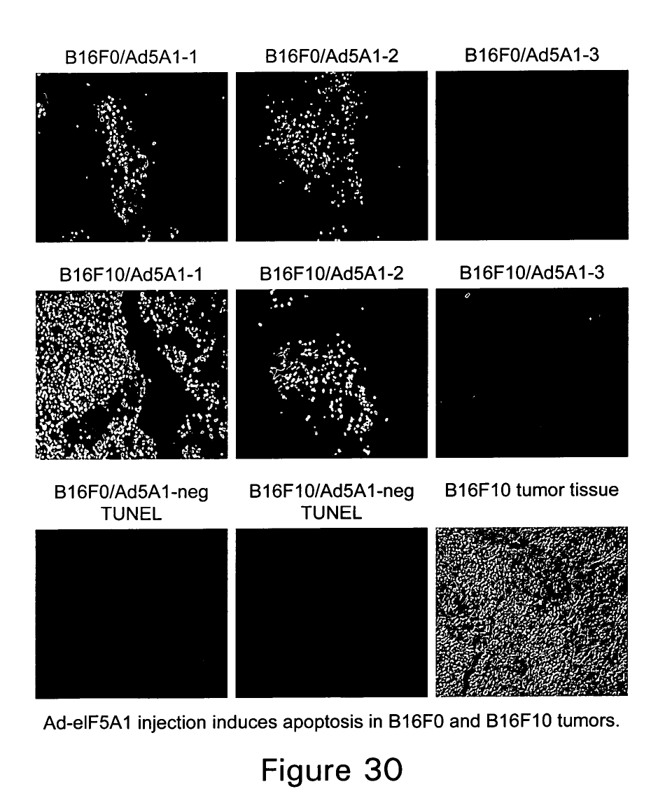

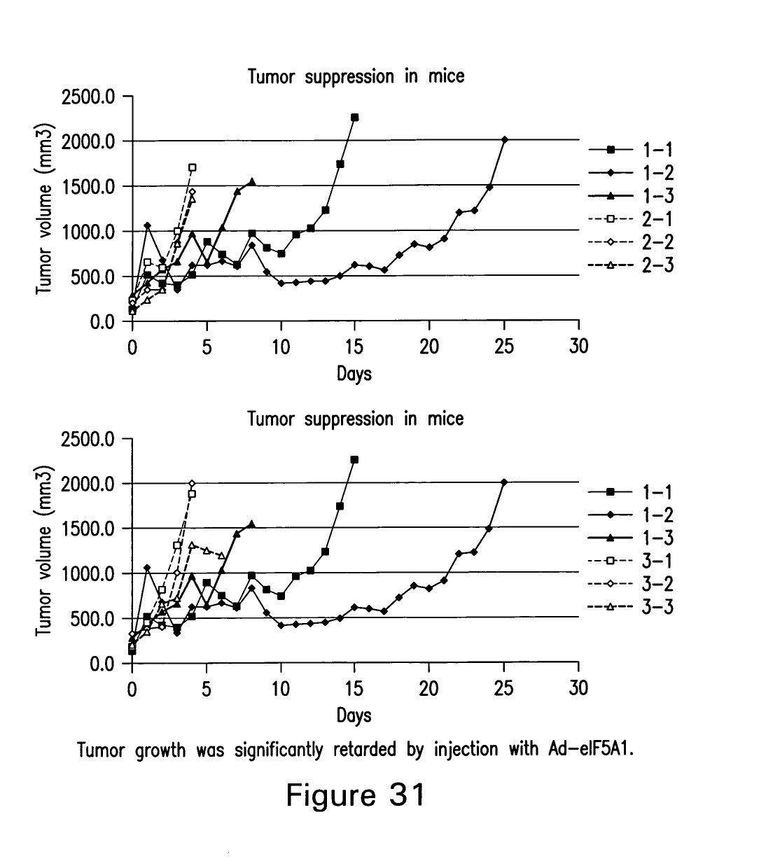

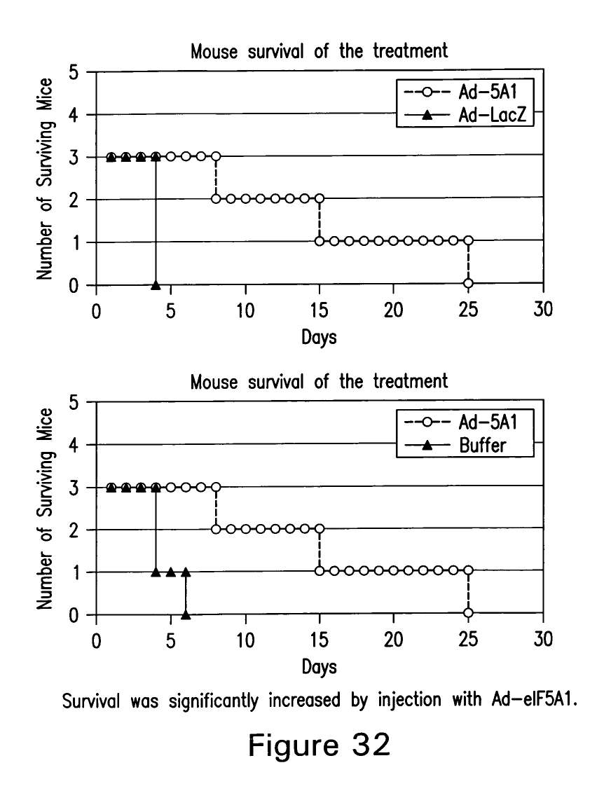

In order to determine whether eIF5A1 treatment induces apoptosis in melanoma tumors, mice bearing either B16F0 or B16F10 subcutaneous tumors were injected intra-tumorally with Ad-eIF5A1 (Experiment IV). Forty-eight hours later the tumors were excised, paraffin embedded and sectioned. TUNEL staining of the sectioned tissues revealed that Ad-eIF5A1 induced apoptosis of tumor cells (FIG. 30). Experiment V was designed to determine whether the ability of intra-tumorally-injected Ad-eIF5A1 to induce apoptosis would result in reduced tumor growth of subcutaneous B16F10 tumors and increase survival. B16F10 cells were injected subcutaneously into the flank of C57BL/6 mice. When the tumors had reached an approximate diameter of 8 mm they were injected intra-tumorally with either Ad-LacZ, Ad-eIF5A1, or buffer. Injections were repeated daily for a total of three days and then every other day thereafter until the tumor exceeded 15 or 16 mm in diameter, at which point the mouse was sacrificed. Tumor dimensions were measure daily using calipers and used to estimate tumor volume. A delay in tumor growth was clearly observed when mice were treated with Ad-eIF5A1 (FIG. 31). Mice that received buffer-only injections lived a maximum of 4 to 6 days after the initiation of treatment while mice that received Ad-LacZ injections only survived 4 days after beginning treatment (FIG. 32). Mice that received Ad-eIF5A1 injections lived at least 8 days after treatment and as much as 25 days after treatment, demonstrating that treatment with Ad-eIF5A1 can result in dramatic improvement in survival of tumor-bearing mice (FIG. 32).

Accordingly, the present invention provides a method of inhibiting cancer growth. The present invention also provides a method of inhibiting or slowing down the ability of a cancer cell to metastasize. Inhibiting cancer growth includes a reduction in the size of a tumor, a decrease in the growth of the tumor, and can also encompass a complete remission of the tumor. Inhibiting cancer growth also means killing cancer cells. The cancer can be any cancer or tumor, including but not limited to colon cancer, colorectal adenocarcinoma, bladder carcinoma, cervical adenocarcinoma, and lung carcinoma.

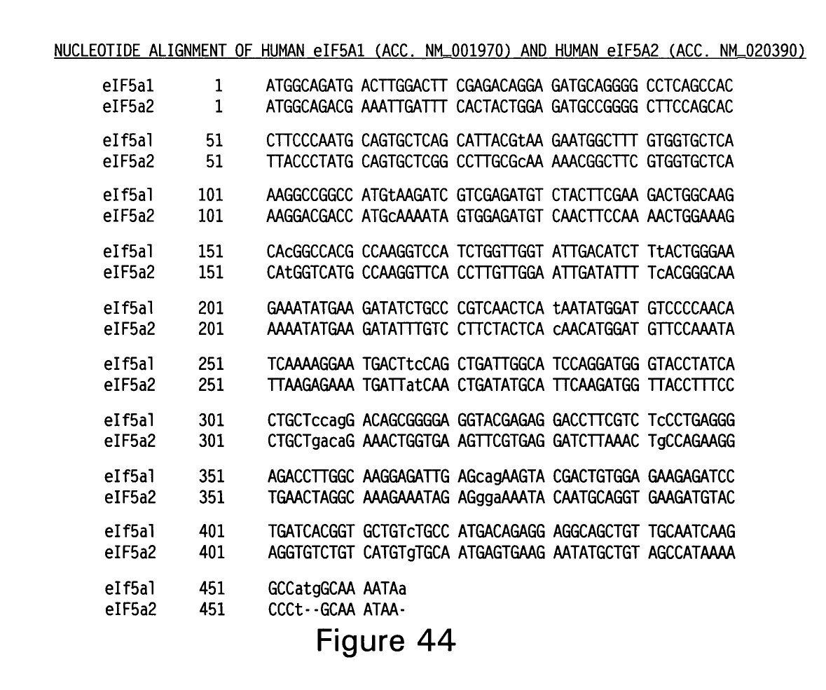

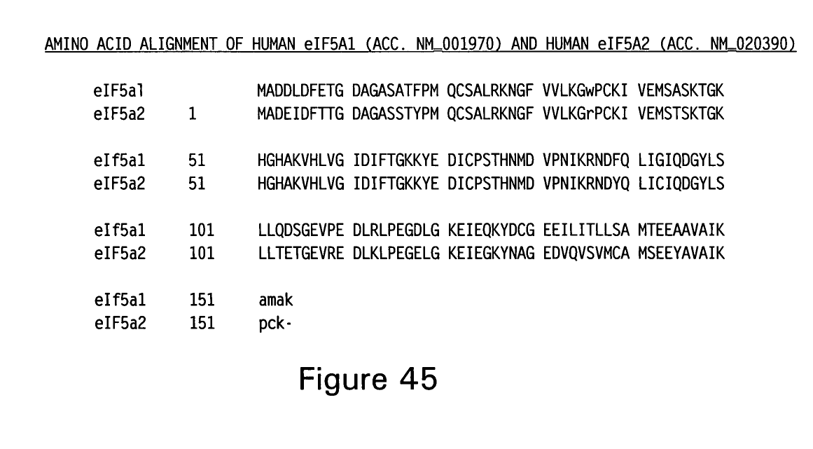

The methods of the present invention involve the administration of a polynucleotide encoding eIF-5A, preferably human eIF-5A1 to a patient (a mammal, preferably a human), preferably eIF-5A1 accession number NM 001970 (See FIG. 44) having said cancer. The eIF-5A2 isoform may also be used (i.e. accession number NM 020390, although eIF-5A1 is preferred. The eIF-5A may be delivered by any suitable method know in the art. It may be delivered as naked DNA, such as DNA in biologically suitable medium and delivered through IV or subcutaneous injection or any other biologically suitable delivery mechanism. Alternatively, the eIF-5A may be delivered in a plasmid, vector such as an adenovirus vector or any suitable expression vector.

Alternatively, the DNA may be delivered in liposomes or any other suitable “carrier” or “vehicle” that provides for delivery of the DNA (or plasmid or expression vector) to the target tumor or cancer cells. See for example, Luo, Dan, et al., Nature Biotechnology, Vol. 18, January 2000, pp. 33-37 for a review of synthetic DNA delivery systems. Although the present inventors have earlier shown that eIF-5A1 is non toxic to normal tissue (see pending application Ser. No. 11/293,391, filed Nov. 28, 2005, which is incorporated herein by reference in its entirety), a delivery system (as compared to direct administration of the eIF5A polynucleotides/plasmid/expression vector) is preferred. A preferred delivery system provides an effective amount of eIF-5A to the tumor or group of cancer cell, as well as preferably provides a targeted delivery to the tumor or group of cancer cells. Thus, it is preferable to deliver the eIF-5A nucleotides/plasmid/expression vector via a vehicle of nanometer size such as liposomes, dendrimers or a similar non-toxic nano-particle. Further, the vehicle preferably protects the eIF-5A nucleotides/plasmid/expression vector from premature clearance or from causing an immune response while delivering an effective amount of the eIF-5A nucleotides/plasmid/expression vector to the tumor or group of cells. Exemplary vehicles may range from a simple nano-particle associated with the eIF-5A nucleotides/plasmid/expression vector to a more complex pegylated vehicle such as a pegylated liposome having a ligand attached to its surface to target a specific cell receptor.

Liposomes and pegylated liposomes are known in the art. In conventional liposomes, the molecules to be delivered (i.e. small drugs, proteins, nucleotides or plasmids) are contained within the central cavity of the liposome. One skilled in the art would appreciate that there are also “stealth,” targeted, and cationic liposomes useful for molecule delivery. See for example, Hortobagyi, Gabriel N., et al., J. Clinical Oncology, Vol. 19, Issue 14 (July) 2001:3422-3433 and Yu, Wei, et al., Nucleic Acids Research. 2004, 32(5);e48. Liposomes can be injected intravenously and can be modified to render their surface more hydrophilic (by adding polyethylene glycol (“pegylated”) to the bilayer, which increases their circulation time in the bloodstream. These are known as “stealth” liposomes and are especially useful as carriers for hydrophilic (water soluble) anticancer drugs such as doxorubicin and mitoxantrone. To further the specific binding properties of a drug carrying liposome to a target cell, such as a tumor cell, specific molecules such as antibodies, proteins, peptides, etc. may be attached on the liposome surface. For example, antibodies to receptors present on cancer cells maybe used to target the liposome to the cancer cell. In the case of targeting multiple myeloma, folate, II-6 or transferrin for example, may be used to target the liposomes to multiple myeloma cells.

Dendrimers are also known in the art and provide a preferable delivery vehicle. See for example Marjoros, Istvan, J., et al, “PAMAM Dendrimer-Based Multifunctional Conjugate for Cancer Therapy: Synthesis, Characterization, and Functionality,” Biomacromolecules, Vol. 7, No. 2, 2006; 572-579, and Majoros, Istvan J., et al., J. Med. Chem, 2005. 48, 5892-5899 for a discussion of dendrimers.

The eIF-5A may also be delivered directly to the site of the tumor. One skilled in the art would be able to determine the dose and length of treatment regimen for delivery of eIF-5A.

Another embodiment of the present invention provides a method of inducing cell death in multiple myeloma cells. Multiple myeloma is a type of bone marrow cancer that produces high levels of inflammatory cytokines, which can lead to bone lesions and tumor progression. Cytokines IL-1B and IL-6 act as growth factors for the myeloma cells.

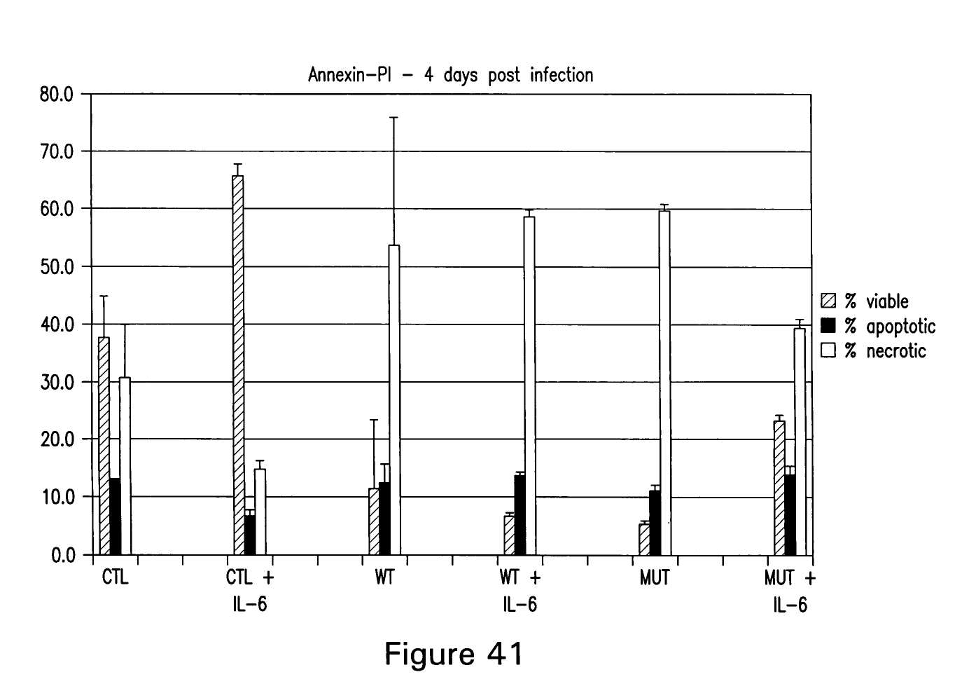

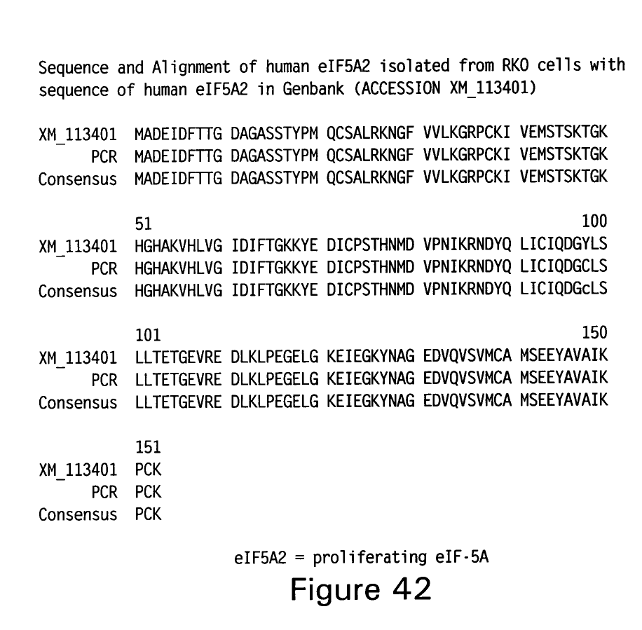

An adenovirus vector construct containing polynucleotides encoding eIF-5A1 (the full length coding region) was administered to a multiple myeloma cell line, KAS 6/1 cells. The KAS 6/1 cell line was created at the Mayo Clinic and reported in Westendorf, J J., et al., Leukemia (1996) 10, 866-876. The cell line was created directly from isolates from a patient with aggressive multiple myeloma. The present inventors have shows that when an adenovirus construct containing eIF-5A is administered to KAS 6/1 cells, there was an increase in the number of dying or dead cells (leaving fewer viable cancer cells)(indicated as “WT” in FIG. 41) as compared to cells having been treated with a control vector alone (indicated as “CTL” in FIG. 1). See FIG. 41. Approximately 90% of the cancer cells treated with Factor 5A1 died, in comparison to approximately 25% of the untreated cells. Accordingly, one embodiment of the present invention provides a method of killing myeloma cells by providing polynucleotides encoding eIF-5A1 to up-regulate expression of eIF-5A1 and cause the multiple myeloma cells to die.

In addition, IL-6 was also administered along with the control (indicated as “CTL+IL-6”) and the eIF-5A construct (indicated as “WT+11-6” in FIG. 41). IL-6 is a cytokine that acts as growth factor for myeloma cells. The results show that even when IL-6 was co-administered with the eIF-5A construct, an increase in apoptosis was still achieved. See FIG. 41. This shows that Factor 5A1 is not only able to kill myeloma cells, but also eliminate myeloma cells in the presence of IL-6. This finding is of interest in that it has proven to be very difficult to induce apoptosis in myeloma cells in the presence of IL-6 with standard therapies such as dexamethasone.

Further, an adenovirus construct with a mutant eIF-5A (K50A) (unable to be hypusinated due to changing the conserved lysine at position 50 to another amino acid) was also administered alone (“MUT”) or with IL-6 (“MUT+IL-6”). The results show that the mutant eIF-5A1 was also able to increase apoptosis as compared to the control cells, even in the presence of IL-6. See FIG. 41. Accordingly, one embodiment of the present invention provides a method of killing myeloma cells by administering a mutant eIF-5A1, said mutant being unable to by hypusinated. The mutant eIF-5A1 causes an increase in expression of unhypusinated eIF-5A1, which increases cell death in the myeloma cells.

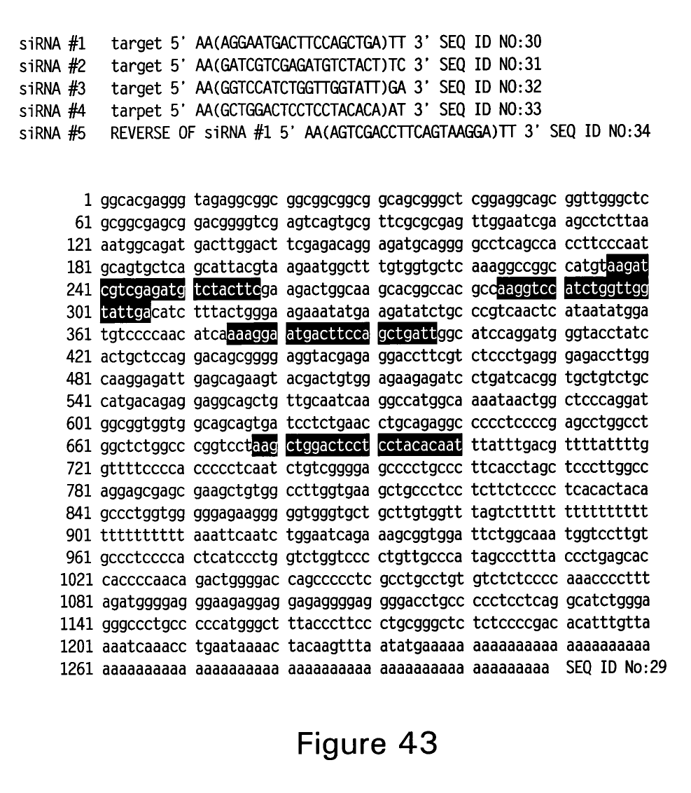

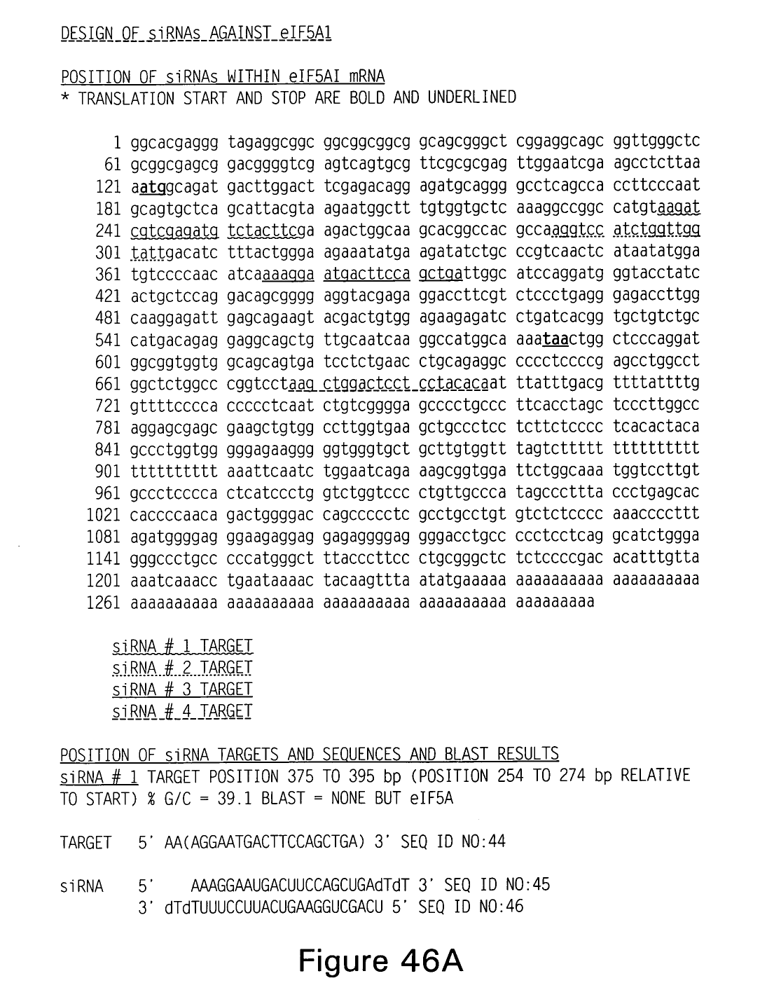

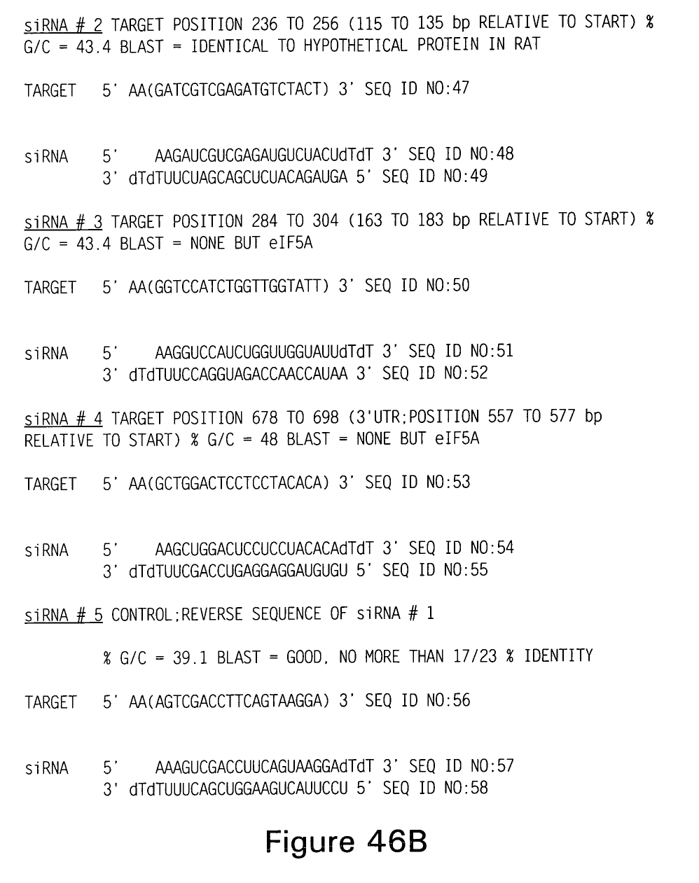

Since IL-6 acts as a growth factor for myeloma cells, down regulating expression of IL-6 would also provide a method of killing myeloma cells. The present inventors have show that siRNA against eIF-5A1 (See FIGS. 43 and 46A and 46B for siRNA constructs) not only down regulates the expression of endogenous eIF-5A1 but also down regulates the expression of various pro-inflammatory cytokines. Thus, one embodiment of the present invention provides a method of killing myeloma cells by administering siRNA against apoptosis-specific eIF-5A to down regulate endogenous expression of IL-1B, which in turn down regulates expression of IL-6, which in turn provides less IL-6 to act as a growth factor for myeloma cells. By down regulating the expression of IL-6, there is less IL-6 available, which is necessary for the continued growth and survival of myeloma cells.

In another embodiment, polynucleotides encoding eIF-5As are administered to provide an increase of apoptosis in the myeloma cells in conjunction with the siRNA against eIF-5A1. The polynucleotides encoding eIF-5A1 are preferably administered in a vector, such as an adenovirus vector, such that the siRNA does not inhibit expression of the exogenous eIF-5A1. For instance, the siRNA targets the 3′ UTR, but the polynucleotides encoding exogenous eIF5A1 preferably contain the entire open reading frame (ORF) and thus have no 3′UTR to be targeted by the siRNA. Suitable siRNA constructs have been previously described in co-pending application Ser. No. 11/287,460; 11/134,445; 11/184,982; and 11/293,391, which are all herein incorporated by reference in their entireties. See also FIGS. 43 and 46A and 46B. The eIF-5A1 is expressed in the myeloma cells and causes cell death. In addition, the eIF-5A1 siRNA decreases expression of endogenous expression of eIF-5A, which in turn decreases expression of IL-6, and in turn increases cell death in the myeloma cells. In this method a mutant eIF-5A as described above may also be used in the vector construct.

The present invention also provides a combination therapy to kill multiple myeloma cells. Compositions comprising polynucleotides encoding eIF5A, preferably eIF5A1 may be administered in conjunction with standard therapies. The eIF5A compositions may be administered before, during or after conventional therapies. The eIF5A may be administered in as a pharmaceutical composition or maybe administered within a delivery vehicle as discussed above.

### EXAMPLES

### Example 1

### In vitro Experiments

Chemicals

N1-guanyl-1,7-diaminoheptane (GC7; Biosearch Technologies), an inhibitor of DHS, was used at a concentration of 50 μM. Actinomycin D (Calbiochem) was used at 0.5 or 1.0 μg/ml. Sodium nitroprusside and desferrioxamine were purchased from Sigma and used at a concentration of 3 mM and 500 μM, respectively. Brefeldin A was also acquired from Sigma and used at a concentration of 4 nM.

Cell Culture and Treatment

The human colon adenocarcinoma cell line, HT-29, was used for cell proliferation and eIF5A localization studies and was a kind gift from Anita Antes (University of Medicine and Dentistry of New Jersey). HT-29 cells were maintained in RPMI 1640 supplemented with 1 mM sodium pyruvate, 10 mM HEPES, and 10% fetal bovine serum (FBS). All other cell lines were obtained from the American Type Culture Collection. CCD112Co is a normal colon fibroblast cell line. RKO is a human colorectal carcinoma cell line (CRL-2577) containing a wild-type p53. The RKO-E6 cell line (CRL-2578) was derived from the RKO cell line. It contains a stably integrated human papilloma virus E6 oncogene and therefore lacks appreciable functional p53 tumor suppressor protein. RKO, RKO-E6, A549, and the cell line CCD112Co, were grown in Modified Eagle Minimum Essential Medium with 2 mM L-glutamine and Earle's Balanced Salt Solution adjusted to contain 1.5 g/L sodium bicarbonate, 0.1 mM non-essential amino acids, 1 mM sodium pyruvate and supplemented with 10% FBS. Cells were maintained at 37° C. in a humidified environment containing 5% CO₂.

Cloning and Construction of Plasmids

Human eIF5A was cloned by RT-PCR from total RNA isolated from RKO cells using the GenElute Mammalian RNA miniprep kit (Sigma) according to the manufacturer's protocol for adherent cells. The primers used were: forward, 5′-CGAGTTGGAATCGAAGCCTC-3′; and reverse, 5′-GGTTCAGAGGATCACTGCTG-3′. The resulting 532 base pair product was subcloned into pGEM-T Easy (Promega) and sequenced. The resulting plasmid was used as a template for PCR using the primers: forward, 5′-GCCAAGCTTAATGGCAGATGATTTGG-3′; and reverse, 5′-CCTGAATTCCAGTTATTTTGCCATGG-3′, and the PCR product was subcloned into the HindIII and EcoRI sites of pHM6 (hemagglutinin [HA] tagged; Roche Molecular Biochemicals) to generate the pHM6-eIF5A vector. A C-terminal truncated construct of eIF5A (pHM6-eIF5AA37) was generated by PCR using the following primers: forward, 5′-GCCAAGCTTAATGGCAGATGATTTGG-3′; and reverse, 5′-GCCGAATTCTCCCTCAGGCAGAGAAG-3′. The resulting PCR product was subcloned into the pHM6 vector. The pHM6-LacZ vector (Roche Molecular Biochemicals) was used to optimize transfection and as a control for the effects of transfection on apoptosis.

Northern Blotting

RKO cells were grown to confluence on 6-well plates and treated for 0, 1, 4, or 8 hours with 1.0 μg/ml Actinomycin D. Total RNA was isolated from the cells using the GenElute Mammalian RNA miniprep kit (Sigma), and 5 μg of RNA was fractionated on a 1.2% agarose/formaldehyde gel. The membrane was probed with a ³²P-labelled cDNA homologous to the 3′-untranslated region (3′-UTR) of eIF5A according to established methods. The eIF5A 3′-UTR cDNA that was used for Northern blotting was cloned by RT-PCR from RKO cells using the following primers: forward, 5′-GAGGAATTCGCTGTTGCAATCAAGGC-3′; and reverse, 5′-TTTAAGCTTTGTGTCGGGGAGAGAGC-3′. The β-actin cDNA that was used as a loading control for Northern blotting was cloned by RT-PCR using the following primers: forward, 5′-GATGATATCGCCGCGCTCGT-3′; and reverse, 5′-GTAGATGGGCACAGTGTGGGTG-3′.

Transfection of Plasmids and Detection of Apoptosis

RKO and RKO-E6 cells were transiently transfected with plasmid DNA using Lipofectamine 2000 (Invitrogen) according to the manufacturer's recommended protocol. Forty-eight hours after transfection, apoptotic cells containing fragmented DNA were detected by terminal deoxynucleotidyl transferase-mediated dUTP-digoxigenin nick end labeling (TUNEL) using a DNA Fragmentation Detection Kit (Oncogene Research Products) according to the manufacturer's protocol. For fluorescence microscopy analysis, cells were transfected on 8-well culture slides, fixed with 4% formaldehyde and then labeled by TUNEL and stained with Hoescht 33258 according to the methods described by Taylor et al. (2004).

Transfection of siRNA

All siRNAs were obtained from Dharmacon. The eIF5A siRNA, which targets the 3′UTR of the eIF5A mRNA (Accession No. BC085015), had the following sequence: sense strand, 5′-GCUGGACUCCUCCUACACAdTdT-3′; and antisense strand, 3′-dTdTCGACCUGAGGAGGAUGUGU-5′. A second siRNA (5A-2) directed against eIF5A had the following sequence: sense strand, 5′-AGGAAUGACUUCCAGCUGAdTdT-3′; and antisense strand, 3′-dTdTUCCUUACUGAAGGUCGACU-5′. The control siRNA that was used had the reverse sequence of the eIF5A-specific siRNA and had no identity to any known human gene product. The control siRNA had the following sequence: sense strand, 5′-ACACAUCCUCCUCAGGUCGdTdT-3′; and antisense strand, 3′-dTdTUGUGUAGGAGGAGUCCAGC-5′. Cells were transfected with siRNA¹² using Lipofectamine 2000 and used in proliferation studies or for Western blotting.

Western Blotting

Protein for Western blotting was isolated using boiling lysis buffer [2% SDS, 50 mM Tris-HCl (pH 7.4)]. Protein concentrations were determined using the Bicinchoninic Acid Kit (Sigma). For Western blotting, 5 μg of total protein was fractionated on a 12% SDS-polyacrylamide gel and transferred to a polyvinylidene difluoride membrane. The primary antibodies used were anti-eIF5A (BD Transduction Laboratories; mouse IgG) and anti-β-actin (Oncogene; mouse IgM), both at a dilution of 1:20,000 in 5% milk. The secondary antibodies were anti-mouse IgG conjugated to horseradish peroxidase (HRP; Sigma) and anti-mouse IgM-HRP (Oncogene). Antibody-protein complexes were visualized using the enhanced chemiluminescence method (ECL, Amersham Biosciences). Following detection of eIF5A, the blots were stripped according to the protocol provided by the ECL Plus Western blotting detection system and reprobed with anti-β-actin antibody to confirm equal loading.

Western blotting of lysate from A549 cells used for MAPK/SAPK pathway analysis was performed using lysate collected in MAPK lysis buffer (10 mM Tris-pH 7.4, 2% SDS, 10% glycerol). Ten micrograms of lysate was separated on 10% SDS-PAGE gels and transferred to a PVDF membrane. The membrane was blocked for one hour in 5% non-fat skim milk in PBS, washed with PBS and incubated with primary antibody at 1:1000 in 5% BSA/PBS-T overnight at 4° C. with shaking. The MAPK/SAPK antibodies (P-p38, p38, P-JNK, JNK, P-ERK, ERK, p90RSK) were purchased from Cell Signaling. The p53 antibodies used in the A549 study were also obtained from Cell Signaling and used in a similar fashion.

2-D Gel Electrophoresis

For 2-D gel electrophoresis, HT-29 cell lysate was harvested in cold lysis buffer (7M Urea, 2M Thiourea, 30 mM Tris, 4% CHAPS, protease inhibitor cocktail), sonicated and cleared of debris by centrifugation. Protein concentration was determined using the Bradford method. The first dimensional isolelectric focusing was performed with the Ettan IPGphor Isoelectric Focusing System (Amersham Biosciences) according to the manufacture's instruction. Immobiline DryStrips (7 cm pH 4-7; Amersham Biosciences) were rehydrated in rehydration buffer (8M Urea, 2% CHAPS, 0.2% DTT, 0.5% pH 4-7 IPG buffer, 0.002% Bromophenol blue) along with cell lysate at room temperature for 12 hours. The isoelectric focusing was performed at 500 V for 30 min, 1000 V for 30 min, and 5000 V for 1 hour and 40 min. The protein on the IPG strip gel was then separated by SDS-PAGE and transferred to a PVDF membrane (Amersham Biosciences). Western blotting was performed using eIF5A antibody (BD Biosciences).

Generation of Adenovirus

Adenoviruses (Adenovirus 5 serotype, E1,E3-deleted) expressing human eIF5A or eIF5A bearing a single point mutation (K50→A50) [eIF5A(K50A)] that prevents hypusination were constructed using the AdMax™ Hi-IQ system (Microbix Biosystems Inc., Toronto, Canada). The site-specific mutation was created in the eIF5A cDNA using PCR. The eIF5A cDNAs were amplified by PCR using plasmid DNA as template and ligated into the SmaI site of the adenovirus shuttle vector pDC516(io). The sequence of the PCR primers were: forward, 5′-GCCAAGCTTAATGGCAGATGATTTGG-3′; and reverse, 5′-CCTGAATTCCAGTTATTTTGCCATGG-3′. The adenovirus genomic plasmid vector pBHGfrt(del)E1,3FLP and the shuttle vectors were propagated in E. coli DH5a and purified using Qiagen EndoFree Plasmid Mega Kit. 5 μg each of the adenovirus genomic plasmid pBHGfrt(del)E1,3FLP and shuttle vector, pDC516(io)-eIF5A or pDC516(io)-eIF5A(K50A), were transfected using the CaCl₂ method recommended by Microbix Biosystems Inc. into 60-80% confluent 293-IQ cells (Microbix Biosystems) in 60 mm culture plates. Plaques appeared after 7 to 10 days incubation at 37° C., and the resulting adenoviral particles [Ad-eIF5A and Ad-eIF5A(K50A)] were amplified in 293-IQ cells. Pure, high titer adenovirus stocks were prepared by CsCl gradient ultracentrifugation according to the protocol provided by Microbix Biosystems Inc. An adenovirus vector expressing LacZ (Ad-LacZ; serotype 5; E1,E3-deleted) was purchased from Qbiogene (California, USA) and employed as a control and reporter in these experiments. The Ad-LacZ adenovirus was amplified and purified in the same manner as the Ad-eIF5A and Ad-eIF5A(K50A) viruses.

Adenovirus Infection and Annexin V Labeling

HT-29, HTB-9, or HTB-4 cells were seeded at 1×10⁶ cells per plate in 100 mm tissue culture plates and infected with adenovirus the following day at 3000 infectious units per cell in 5 ml of RPMI 1640+2% FBS. Additional media was added to the cells after four hours and the concentration of FBS brought to 10%. Twenty-four, forty-eight, or seventy-two hours after infection the cells were detached by trypsinization, washed and stained with Annexin V-FITC according to the manufacturers' protocol (BD Biosciences). The cells were sorted by flow cytometry (Coulter Epics XL-MCL) with a 488 nm argon laser source and filters for fluorescein detection and the data analyzed by WinMDI 2.8.

HT-29 cells were infected with 3000 infectious units per cell and experiments with A549 cells were performed using 1500 infectious units per cell.

Proliferation Assays

HT-29 cells were transfected with siRNA on 96-well plates using Lipofectamine 2000 (Invitrogen). Metabolic activity of proliferating cells was measured with the XTT Cell Proliferation Kit (Roche Applied Science). The BrdU Cell Proliferation Kit (Roche Applied Science) was used to measure DNA synthesis following the manufacturer's protocol. For XTT assays performed following adenovirus infection, 5000 cells were plated per well in a 96-well plate. The next day cells were infected with Ad-lacZ, Ad-eIF5A1 and Ad-eIF5A1(K50A) (Ad-eIF5A1M) respectively with untreated cells as negative control and Actinomycin D treated cell as positive control. XTT substrate was added and A475 nm was measured with A690 nm as the reference.

Indirect Immunofluorescence

HT-29 cells were cultured on poly-L-lysine-coated glass coverslips. Subconfluent cells were incubated for 16 hours with 200 Units of interferon gamma (IFN-γ; Roche Applied Science) followed by TNF-α (100 ng/ml; Leinco Technologies) for times varying from 10 minutes to 8 hours. Alternatively, cells were treated with 1.0 μg/ml Actinomycin D for increasing lengths of time from 30 minutes to 16 hours. The treated cells were fixed with 3% formaldehyde (methanol-free; Polysciences Inc.) for 20 minutes, washed twice for 5 minutes with PBS and once for 5 minutes with PBS containing 100 mM glycine, and permeabilized with 0.2% Triton X-100 in PBS for 4 minutes. Cells were then labeled for immunofluorescence using a standard protocol. The primary antibody was anti-eIF5A (BD Transduction Laboratories; mouse IgG) incubated at a dilution of 1:250 for 1 hour. The secondary antibody was anti-mouse IgG-AlexaFluor 488 (Molecular Probes) used at a dilution of 1:200 for 1 hour. Following antibody labeling, the nuclei were stained with Hoescht 33258, and the labeled cells were observed by fluorescent microscopy.

Matrige™ Cell Invasion Assays

A549 cells were infected with adenovirus at 1500 infectious units per cell and incubated for 24 hours. The cells were then detached with trypsin, washed with serum-free media, and plated in serum-free media at 30,000 cells per well on a transwell (Falcon 8.0 μm cell culture insert) that had been precoated with 15 μg of Matrigel™ Basement Membrane Matrix (BD Biosciences). Media containing 10% FBS was placed in the bottom well (the well of the 24-well plate in which the transwell is resting) and the cells were incubated for a further 24 hours. After the incubation, the media was removed from the upper chamber of the transwell, the transwell was removed and placed into the well of a 24-well plate containing 500 microliters of crystal violet. The transwell was incubated 20 minutes in the dye and then washed repeatedly by dunking the transwell in a beaker of water. A pre-wetted cotton swab was used to scrape cells from the top surface of the transwell. Cells that had migrated to the bottom surface of the transwell were viewed by light microscopy, photographed, and the number of migrated cells per field were counted. See FIG. 24.

Statistics

Student's t-test was used for statistical analysis. Significance was determined by a confidence level above 95% (P<0.05).

### Example 2

### In vivo Experiments

Mice and Establishment of Tumors

C57BL/6 mice were purchased from Charles River, Quebec, Canada at 5-7 weeks of age. Mice were allowed one week to acclimate before experimentation began. B16F10 murine melanoma cells were purchased from ATCC and cultured in DMEM-10% FBS. The cell monolayer was trypsinized and neutralized with MEM-10% FBS. Cells were washed with PBS twice and cell viability was determined by trypan blue staining. For experimental metastasis experiments (Experiments II and III), melanoma tumors were established in the lung by tail vein injection of B16F10 cells into 6-week old mice. B16F10 cells were diluted to 1×10⁶ viable cells/ml in PBS. 200 ul of cells was injected into each mouse via tail vein. For subcutaneous tumor experiments (Experiments IV and V), melanoma tumors were established by subcutaneous injection of 500,000 B16F10 cells into the right flank of 10 to 14-week old mice. At the end of all experiment (when mice became moribund or tumor exceeded a pre-determined size), the mice were euthanized by CO₂ inhalation.

### Example 3

### Experiment II

Construction and Purification of Plasmid DNA

The pCpG-lacZ expression vector lacking CpG dinucleotides was purchased from InvivoGen, San Diego, USA. An HA-tagged eIF5A1 cDNA was subcloned into the pCpG-lacZ vector by first digesting the plasmid with NcoI and NheI and isolating a 3.1 kb of pCpG vector backbone (thereby removing LacZ coding sequence) and ligating with a PCR amplified cDNA of eIF5A1 containing an HA tag (pCpG-HA5A1). The PCR primers were eIF5A1 for: HA-5A1 for: 5′-GCTCCATGGATGTACCCATACGACGTCCC-3′; and eIF5A1 rev: 5′-CGCGCTAGCCAGTTATTTTGCCATCGCC-3′. The pCpG-lacZ and pCpG-HA5A1 were amplified in E. coli GT115 cultured in LB or 2XYT medium containing 25 μg/ml of zeocin.

The plasmids were extracted and purified by QIAGEN Endofree Plasmid Giga kit. The DNA concentration was measured by UV absorption at 260 nm and agarose gel electrophoresis.

Tail Vein Injection of Plasmid DNA (Experiment II):

Plasmid DNA dissolved in 1×PBS (around 200 μl based on body weight) were injected into the tail vein of mice at days 2, 4, 7, 11, 16, 21, 26, 31. Plasmid DNA concentration was 660 ng/μl for 2× (6.6 mg/kg), 330 ng/μl for 1× (3.3 mg/kg), and 33 ng/μl for 0.1× (0.33 mg/kg).

Body and Lung Weights:

Body weights were measured before tail vein injection or every Monday and Friday. Mice were euthanized with CO₂ when they reached morbidity (lethargic, respiratory distress) and lungs were removed, weighed, photographed, frozen, and stored at −70° C. See FIGS. 25-27.

VEGF Elisa:

Harvested lung tissues were washed with PBS, frozen in dry ice, and stored at −70° C. Protein lysates were isolated from ground lung tissues. 50 μg of lung tissue proteins were used to determine VEGF concentration in mouse lung using Mouse VEGF Immunoassay kit (R&D Systems, Inc. Minneapolis, USA).

### Example 4

### Experiment III

Injection of Plasmid DNA/DOTAP Complexes

6 weeks old mice were tail vein injected with B16F10 melanoma cells (50,000 cells in 200 μl PBS/mouse), plasmid DNA, and DNA carrier complex containing 50 μg of endotoxin-free kit purified plasmid DNA, and 80 μg of DOTAP were tail vein injected into mice at days 7, 14, and 21. Mice were sacrificed at day 25. Lungs were removed, weighed, and photographed. See FIGS. 28-29.

### Example 5

### Experiment IV

Injection of Adenovirus Constructs and TUNEL

10-week old C57BL/6 mice were injected subcutaneously into the right flank with either 500,000 B16F0 or B16F10 cells. 1×10⁹ pfu Ad-5A1 in 50 ul of PBS was injected into tumors when the tumors reached about 4 mm in diameter (10-12 days after B16 cell injection). Mice were sacrificed after 48 hours and tumors were excised, fixed, and embedded in paraffin. Two sections for each cell tumor type (Ad-5A1-1 and Ad-5A1-2) were stained by TUNEL (Promega) according to the manufacturer's protocol. Negative control slides (Ad-5A1-neg) in which the TdT enzyme was left out of the TUNEL reaction were included for each cell type. See FIG. 30.

### Example 6

### Experiment V

Establishment of Tumors

14-week old C57BL/6 mice were injected subcutaneously with 500,000 B16F10 cells (in 100 ul of PBS) on the right flank. The progress of tumor formation was checked daily until the tumor size reached around 8 mm in diameter.

Injection with Adenovirus

Treatment began when tumor size reached a diameter of 8 mm. Mice were injected with either 1×10⁹ plaque forming units (pfu) of Ad-eIF5A1 or Ad-LacZ. Injections were distributed over three sites in the tumor. Mice were injected every day for the first three days and then every other day thereafter until the mouse was sacrificed. Mice were sacrificed when the tumor size exceeded 15 to 16 mm in one dimension. Buffer only mice received only the buffer in which the adenovirus was suspended (10 mM Tris-HCl pH 7.4, 10 mM MgCl₂, 10% glycerol). The tumor dimensions were measured every day using calipers and the tumor volume was calculated using the equation below:
Tumor volume (mm³)=L*W²*0.52

Where L=length, W=width (always shorter dimension) See FIGS. 31 and 32.

### Example 7

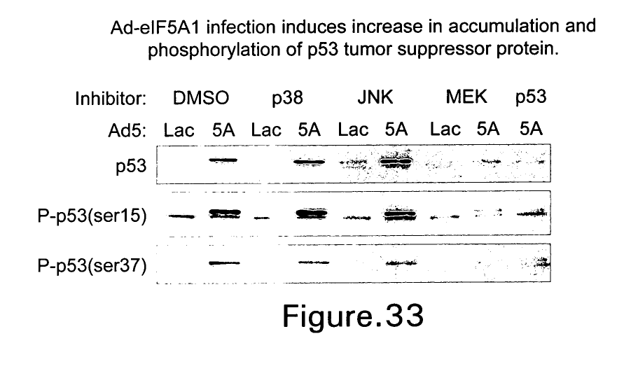

A549 lung carcinoma cells were infected with either an adenovirus expressing LacZ (Lac) or eIF5A1 (5A). Four hours after infection, the media was replaced with media containing either DMSO, 10 μM of the p38 inhibitor SB203580 (Calbiochem), 10 μM of the JNK inhibitor II (Calbiochem), 10 μM of the MEK inhibitor U1026 (Calbiochem), or 30 μM of the p53 inhibitor Pifithrin-a (Calbiochem). Forty-eight hours later, the cells were treated with EGF for 30 minutes and the cell lysate was harvested. Western blots were performed on the lysate using antibodies directed against either total p53 (p53), or p53 phosphorylated on serine 15 [P-p53(ser15)], or p53 phosphorylated on 37 [P-p53(ser37)]. See FIG. 33.

The results shown in FIG. 33 shows that Ad-eIF5A1 induces accumulation of p53 by 48 hours after infection. The figure also shows that Ad-eIF5A1 induces phosphorylation of p53 by 48 hours after infection; accumulation and phosphorylation of p53 is inhibited by inhibitor of MEK; accumulation and phosphorylation of p53 is inhibited by inhibitor of p53 activity; eIF5A1 stimulates MEK-dependent phosphorylation of p53; and eIF5A1 stimulates p53-dependent accumulation of p53.

### Example 8

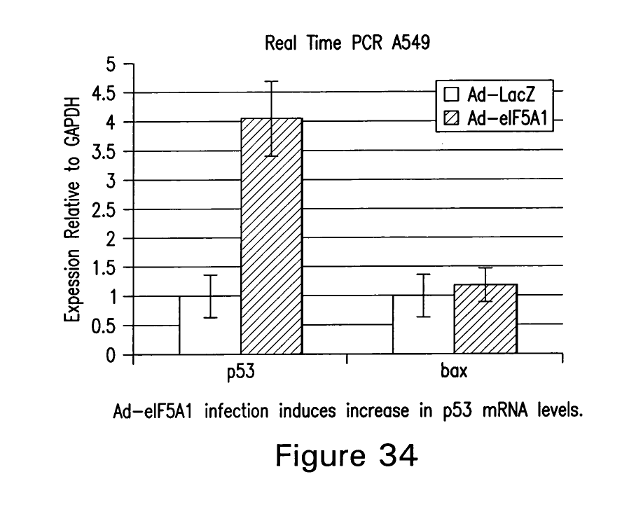

A549 lung carcinoma cells were infected with either an adenovirus expressing LacZ (Ad-LacZ) or eIF5A1 (Ad-eIF5A1). Forty-eight hours later, the total RNA was isolated from the cells. The levels of p53 and bax mRNA transcript levels were determined by Real Time PCR using GAPDH as a reference gene. The p53 primers were: 5′-CGCTGCTCAGATAGCGATGGTC-3′ (5′-primer) and 5′-CTTCTTTGGCTGGGGAGAGGAG-3′ (3′-primer) [These p53 primer sequences were obtained from: Li et al. (2004). A Novel eIF5A/complex Functions as a Regulator of p53 and p53-dependent Apoptosis, J Biol. Chem. 279 49251-49258]. See FIG. 34, which shows that over-expression of eIF5A1 induces accumulation of p53 at mRNA level, yet no effect on bax was seen.

### Example 9

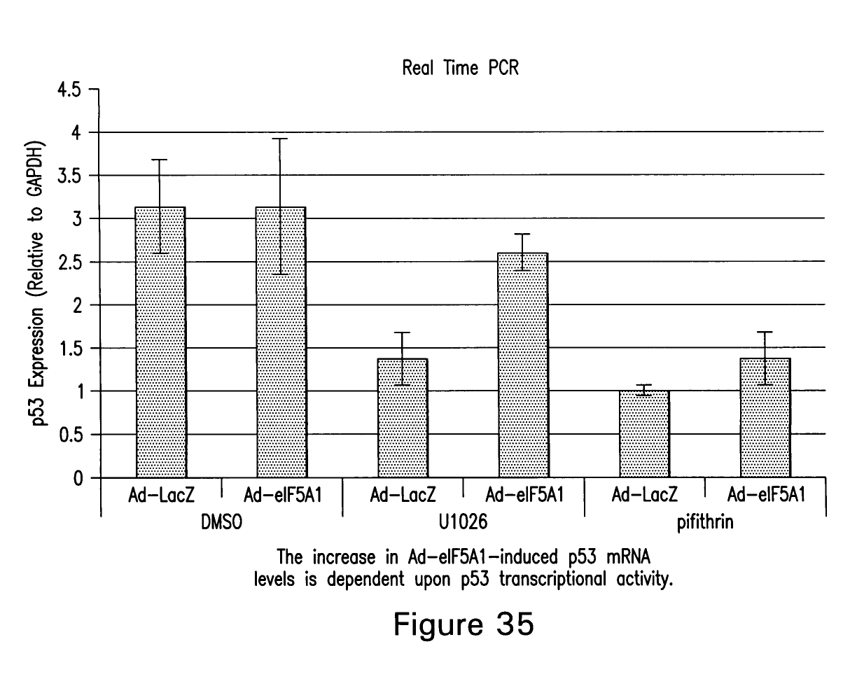

A549 lung carcinoma cells were infected with either an adenovirus expressing LacZ (Ad-LacZ) or eIF5A1 (Ad-eIF5A1). Four hours after infection, the media was replaced with media containing either DMSO, 10 μM of the MEK inhibitor U1026 (Calbiochem), or 30 μM of the p53 inhibitor Pifithrin-a (Calbiochem). Forty-eight hours later, the total RNA was isolated from the cells. The levels of p53 transcript levels were determined by Real Time PCR using GAPDH as a reference gene. The p53 primers were: 5′-CGCTGCTCAGATAGCGATGGTC-3′ (5′-primer) and 5′-CTTCTTTGGCTGGGGAGAGGAG-3′ (3′-primer) [These p53 primer sequences were obtained from: Li et al. (2004). A Novel eIF5A/complex Functions as a Regulator of p53 and p53-dependent Apoptosis, J Biol. Chem. 279 49251-49258]. See FIG. 35, which shows that the eIF5A1-dependent accumulation of p53 is dependent on p53 transcriptional activity, and the upregulation of p53 protein that occurs in response to eIF5A1 results in increased transcription of p53 mRNA.

### Example 10

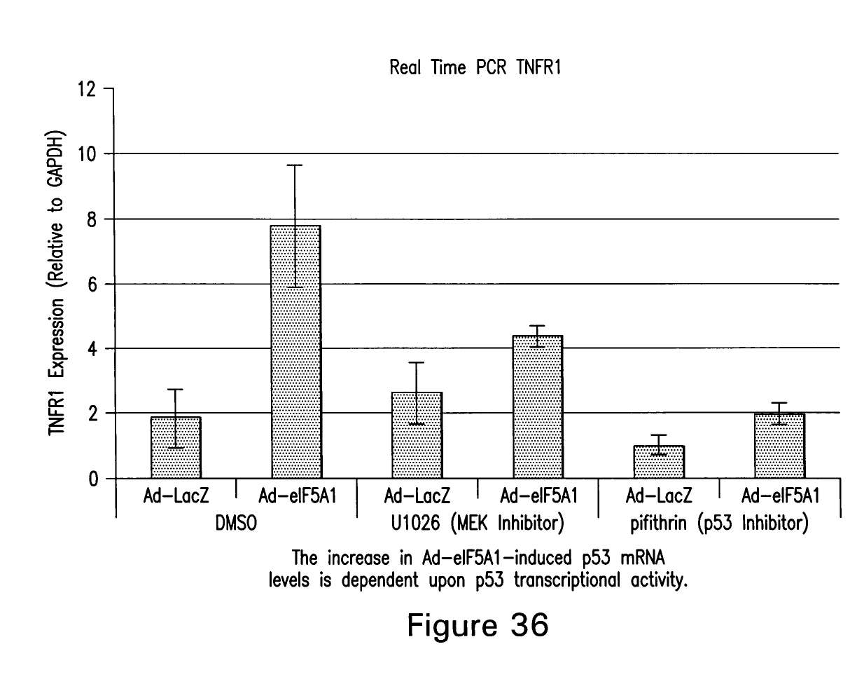

A549 lung carcinoma cells were infected with either an adenovirus expressing LacZ (Ad-LacZ) or eIF5A1 (Ad-eIF5A1). Four hours after infection, the media was replaced with media containing either DMSO, 10 μM of the MEK inhibitor U1026 (Calbiochem), or 30 μM of the p53 inhibitor Pifithrin-a (Calbiochem). Forty-eight hours later, the total RNA was isolated from the cells. The levels of p53 transcript levels were determined by Real Time PCR using GAPDH as a reference gene. The TNFR1 primers were: TNFR1-F 5′ ATCTCTTCTTGCACAGTGG 3′ and TNFR1-R 5′ CAATGGAGTAGAGCTTGGAC 3′. See FIG. 36, which shows that TNFR1 mRNA levels are upregulated by infection with Ad-eIF5A1 and that this accumulation of TNFR1 mRNA is partially dependent on MEK. This accumulation of TNFR1 mRNA is dependent on p53 transcriptional activity.

### Example 11

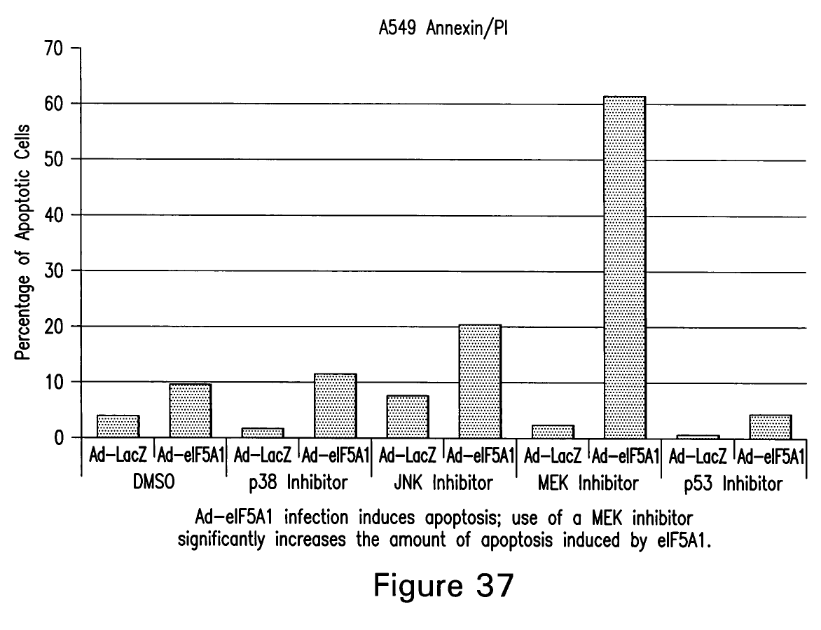

A549 lung carcinoma cells were infected with either an adenovirus expressing LacZ (Lac) or eIF5A1 (5A). Four hours after infection, the media was replaced with media containing either DMSO, 10 μM of the p38 inhibitor SB203580 (Calbiochem), 10 μM of the JNK inhibitor II (Calbiochem), 10 μM of the MEK inhibitor U1026 (Calbiochem), or 30 μM of the p53 inhibitor Pifithrin-a (Calbiochem). Forty-eight hours later, the cells were harvested and the percentage of cells undergoing apoptosis was determined by Annexin/PI staining (BD Bioscience) followed by analysis by flow cytometry. See FIG. 37.

FIG. 37 shows that Ad-eIF5A1 induces apoptosis by 48 hours after infection; that inhibition of JNK increases apoptosis induced by eIF5A1; that inhibition of MEK increases apoptosis induced by eIF5A1, and that inhibition of p53 decreases apoptosis induced by eIF5A1.

### Example 12

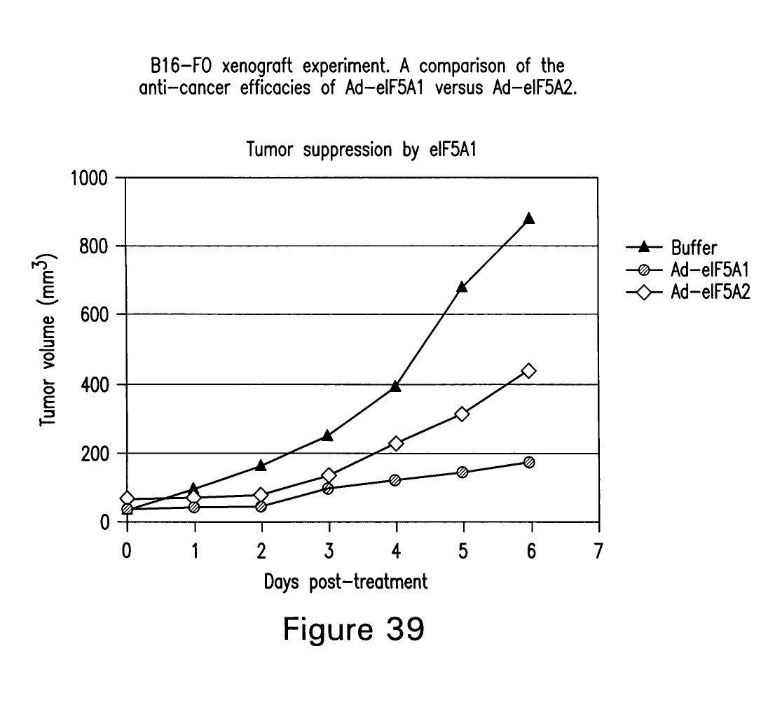

Mice were injected with 50,000 B16-F0 melanoma cells sub-cutaneously. When the tumors reached a size of around 5×5 mm (65 mm³) intra-tumoral injections were initiated. 1×10⁹ pfu of either Ad-lacZ (group 2), Ad-eIF5A1 (group 3), or Ad-eIF5A2 (group 4) diluted in 50-100 μl of PBS/10% glycerol buffer or buffer only (group 1) were injected into the tumors in three sites per tumor every other day. The tumor size was measured every other day until every the other day until the sacrifice of mice when tumor size reached 10% of the body weight. See FIG. 39.

### Example 13

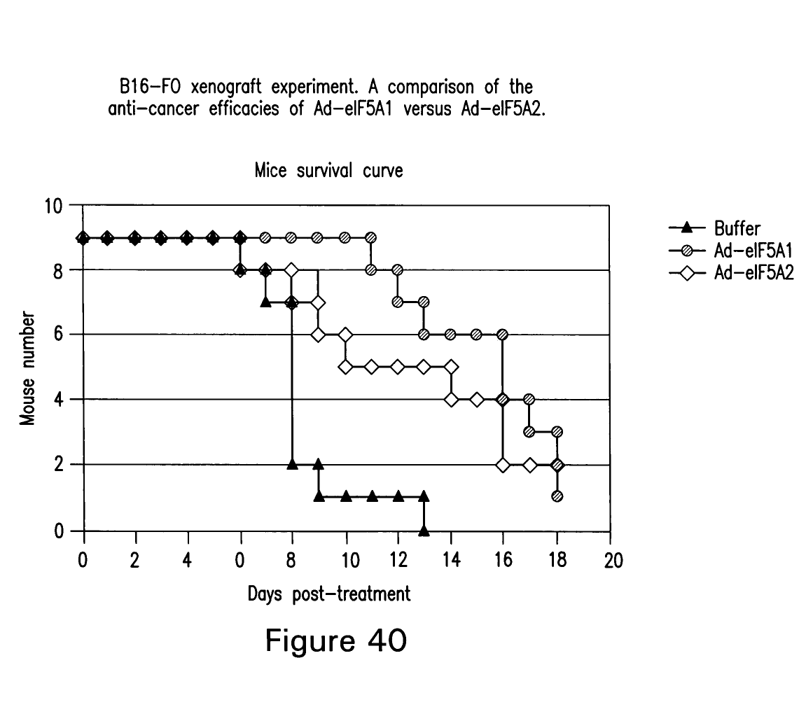

Mice were injected with 50,000 B16-F0 melanoma cells sub-cutaneously. When the tumors reached a size of around 5×5 mm (65 mm³) intra-tumoral injections were initiated. 1×10⁹ pfu of either Ad-lacZ (group 2), Ad-eIF5A1 (group 3), or Ad-eIF5A2 (group 4) diluted in 50-100 μl of PBS/10% glycerol buffer or buffer only (group 1) were injected into the tumors in three sites per tumor every other day. The mice were sacrificed when tumor size reached 10% of body weight. See FIG. 40

### Example 14

### Multiple Myeloma

On day 0, KAS 6/1 multiple myeloma cells were infected with 3000 IFU/cell wild-type or mutant eIF5a (unable to be hypusinated—conserved lysine is mutated) adenovirus vector construct for 4 hours. Three replicates with or without IL-6 present in the post-infection culture media were set up. In addition KAS cells were plated for controls (were not infected). On day 2 and 4, MTT and annexin/PI assays were performed. The supernatants were harvested. The results are shown in FIG. 41.

1. A composition for killing myeloma cells comprising a polynucleotide encoding eIF5A.

2. The composition of claim 1 wherein the eIF5A is selected from the group consisting of eIF5A1, eIF5A2 or a mutant eIF5A1.

3. The composition of claim 2 wherein the eIF5A is eIFA1.

4. The composition of claim 3 wherein the composition further comprises a delivery vehicle.

5. The composition of claim 4 wherein the delivery vehicle is selected from the group consisting of a vector, plasmid, liposome, or dendrimer.

6. The composition of claim 5 wherein the delivery vehicle is a vector.

7. The composition of claim 6 wherein the delivery vehicle is an adenovirus vector.

8. The composition of claim 5 wherein the delivery vehicle is a liposome.

9. The composition of claim 5 wherein the delivery vehicle is a dendrimer.

10. Use of eIFA to make a medicament to kill multiple myeloma cells in a subject having multiple myeloma.

11. The use of eIF5A of claim 10 wherein the eIF5A is eIF5A1, eIF5A2, or a mutant eIF5A1 wherein the mutant eIF5A1 has had the conserved lysine changed to an alanine or any other amino acid, and wherein the mutant is unable to be hypusinated.

12. A method of killing multiple myeloma cells, the method comprising administering to the myeloma cells a composition comprising a polynucleotide encoding eIF5A1, wherein the composition kills the multiple myeloma cells.

13. The method of claim 12 wherein the eIF5A1 is a mutant, wherein said mutant has had the conserved lysine changed to an alanine or any another amino acid and wherein said mutant is unable to be hypusinated.

14. The method of claim 12 wherein the composition comprises a vector.

15. The method of claim 14 wherein the vector is an adenovirus vector.

16. The method of claim 12 further comprising administering siRNA directed against eIF-5A1, wherein said siRNA down regulates endogenous expression of eIF-5A1, and wherein said down-regulation of expression of eIF-5A1 down regulates expression of IL-6 and wherein said down regulation of IL-6 kills multiple myeloma cells.

17. A method of inducing apoptosis in multiple myeloma cells in a subject having multiple myeloma, said method comprising administering the composition of claim 3, wherein the eIF5A1 in said composition induces apoptosis in the multiple myeloma cells.

18. The method of claim 17 wherein the composition is administered intravenously.

19. The method of claim 17 wherein the composition further comprises a liposome.

20. The method of claim 17 wherein the composition further comprises a dendrimer.

21. A method of killing multiple myeloma cells comprising administering the composition of claim 3 and further administering a conventional multiple myeloma therapy.
